(19) United States
(12) Patent Application Publication
(10) Pub. No.: US 2022/0251105 A1
(43) Pub. Date: Aug. 11, 2022

(54) METHODS OF TREATING A MK2-MEDIATED DISORDER

(71) Applicant: Celgene CAR LLC, Pembroke (BM)

(72) Inventor: Francisco Ramirez-Valle, New York, NY (US)

(21) Appl. No.: 17/611,594

(22) PCT Filed: May 15, 2020
(86) PCT No.: PCT/US20/33232

§ 371 Date: Nov. 16, 2021

Related U.S. Application Data
(60) Provisional application No. 62/849,185, filed on May 17, 2019.

(51) Int. Cl. C07D 495/14

(52) U.S. Cl. CPC C07D 495/14

(57) ABSTRACT

The present invention provides methods of treating, stabilizing or lessening the severity or progression of one or more diseases or conditions associated with MK2.

### CROSS REFERENCE TO RELATED APPLICATIONS

The present application claims priority to U.S. Provisional Application No. 62/849,185, filed May 17, 2019, the entirety of which is hereby incorporated by reference.

### SUMMARY

In some embodiments, the present invention provides methods of treating, stabilizing or lessening the severity or progression of one or more diseases and conditions associated with mitogen-activated protein (MAP) kinase-activated protein kinase 2 (MAPKAPK2; MK2). In some aspects, the present invention provides methods of treating, stabilizing or lessening the severity or progression of one or more diseases and conditions associated with MK2 comprising administering to a patient in need thereof a pharmaceutically acceptable composition comprising

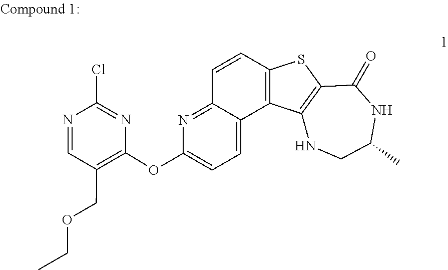 [SMILES: CCOCc1cnc(Cl)nc1Oc1ccc2c(ccc3sc4c(c32)NC[C@@H](C)NC4=O)n1]

or a pharmaceutically acceptable salt thereof. It will be appreciated that references to “Compound 1” below include all forms of Compound 1, including the free base form of Compound 1, a pharmaceutically acceptable salt of Compound 1, a crystal or solid form of Compound 1, and non-covalent complexes comprising Compound 1 and a co-former.

Compound 1 is a potent, covalent, and irreversible inhibitor of MK2 in both biochemical (IC₅₀ of 156.3±5.5 nM) and cell based assays (EC₅₀ of 89±2.6 nM utilizing inhibition of Hsp27 phosphorylation as the proximal readout of activity). Covalent interaction with MK2 was confirmed in cell-based assays and in vivo from PBMC isolated in rodent pharmacology studies. In multiple models of rodent disease, including a rodent model of ankylosing spondylitis, Compound 1 demonstrated reduction in disease scores. These data support the potential benefit of MK2 inhibition by Compound 1 in arthropathies and possibly other inflammatory diseases in humans. Accordingly, in some embodiments, the present invention provides a method of treating, stabilizing or lessening the severity or progression of one or more diseases or disorders associated with MK2 selected from group consisting of ankylosing spondylitis, rheumatoid arthritis, psoriatic arthritis and psoriasis. In some such embodiments, the method comprises administering to a patient in need thereof a pharmaceutically acceptable composition comprising Compound 1.

In some embodiments, provided methods comprise orally administering to a patient compositions comprising Compound 1. In some embodiments, such compositions are capsule formulations. In general, provided methods comprise administering a composition which comprises Compound 1 and one or more pharmaceutically acceptable excipients, such as, for example, binders, diluents, disintegrants, wetting agents, lubricants and adsorbents.

In some embodiments, the present invention also provides dosing regimens and protocols for the administration of Compound 1 to patients in need thereof.

### BRIEF DESCRIPTION OF THE DRAWINGS

FIG. 1 depicts the effect of Compound 1 on arthritis disease scores in Male HLA-B27/Huβ2m rats. FIG. 1A depicts the differences between treatment groups at designated time points were analyzed using two-way analysis of variance (ANOVA) and Dunnett's post hoc. Error bars represent standard error of the mean (SEM). FIG. 1B depicts the statistical significance vs vehicle. FIG. 1C depicts the differences between treatment groups were also determined by calculating AUC of clinical scores and analyzing data by ANOVA with Kruskal Wallis post hoc. Error bars represent SEM.

### DETAILED DESCRIPTION OF THE INVENTION

### Definitions

As used herein, the term “about”, when used in reference to a numerical value, means±10% of that value. For example, a dose that comprises “about 100 mg” of Compound 1 encompasses any amount of Compound 1 within a range of 90 mg to 110 mg.

As used herein, “disease(s) or disorder(s) associated with MK2” means any disease or other deleterious condition in which mitogen-activated protein (MAP) kinase-activated protein kinase 2 (MAPKAPK2; MK2), or a mutant thereof, is known or suspected to play a role. Accordingly, another embodiment of the present invention relates to preventing, treating, stabilizing or lessening the severity or progression of one or more diseases in which MK2, or a mutant thereof, is known or suspected to play a role. Specifically, the present invention relates to a method of treating or lessening the severity of a proliferative disorder, wherein said method comprises administering to a patient in need thereof Compound 1 or a pharmaceutically acceptable composition thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a compound, or composition containing a compound, which is sufficient for treating one or more symptoms of a disorder or condition associated with MK2.

The term “subject”, as used herein, means a mammal and includes human and animal subjects, such as domestic animals (e.g., horses, dogs, cats, etc.). It will be appreciated that the term “subject” is sometimes used synonymously with “patient.”

The terms “treat” or “treating,” as used herein, refers to partially or completely alleviating, inhibiting, delaying onset of, preventing, ameliorating and/or relieving a disorder or condition, or one or more symptoms of the disorder or condition. As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The expression “unit dosage form” as used herein refers to a physically discrete unit of inventive formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

### The Role of MK2 in Human Disease

MAP kinase-activated protein kinase 2 (“MK2”) is an enzyme that is encoded by the human MAPKAPK2 gene. The MK2 enzyme is a serine/threonine (Ser/Thr) protein kinase that is regulated through direct phosphorylation by p38 MAP kinase.

MK2 is a multi-domain protein consisting of an N-terminal proline-rich domain, a catalytic domain, an autoinhibitory domain and at the C-terminus a nuclear export signal (NES) and nuclear localization signal (NLS). Two isoforms of human MK2 have been characterized. One isoform consists of 400 amino acids and the other isoform consists of 370 residues which is thought to be a splice variant missing the C-terminal NLS.

MK2 is known to be involved in many cellular processes including stress and inflammatory responses, nuclear export, gene expression regulation and cell proliferation. Indeed, MK2 regulates, by a post-transcriptional mechanism, biosynthesis of tumor necrosis factor alpha (TNF-α) that is overproduced in inflammatory diseases such as rheumatoid arthritis and inflammatory bowel disease. See Natesan et al., J. Med. Chem. 2012, 55, 2035-2047.

MK2 inhibitors prevent or block phosphorylation of heat shock protein 27 (Hsp27). Inhibition of Hsp27 phosphorylation occurs by inhibiting the formation of the p38 kinase-MK2-Hsp27 signaling complex. Phosphorylation of Hsp27 is the penultimate event in a complex signaling cascade that occurs in response to extracellular stimuli. See Zheng et al., The Journal of Biological Chemistry, vol. 281, no. 48, 37215-37226, Dec. 1, 2006. Hsp27 usually exists as oligomers and plays a role in regulation of many cellular functions such as inhibition of the death receptor-mediated apoptosis, promotion of proper refolding of denatured proteins by acting as a molecular chaperone, and regulation of cytoskeleton. The presence of MK2 is a necessary condition for the formation of p38 kinase-MK2-Hsp27 signaling complex in cells. See Zheng et al., The Journal of Biological Chemistry, vol. 281, no. 48, 37215-37226, Dec. 1, 2006.

Evidence suggests that many signaling proteins form multimeric complexes. See Zheng et al., The Journal of Biological Chemistry, vol. 281, no. 48, 37215-37226, Dec. 1, 2006. One such complex is the Hsp27/Akt (a serine/threonine kinase) dimer, which forms in the cytoplasm of a cell. Another complex is formed between MK2 and p38. See Ben-Levy et al., Current Biology 1998, 8:1049-1057; Natesan et al., J. Med. Chem. 2012, 55, 2035-2047; Zheng et al., The Journal of Biological Chemistry, vol. 281, no. 48, 37215-37226, Dec. 1, 2006.

In unstimulated conditions, inactive p38 and unphosphorylated MK2 form such dimer in the nucleus of a cell. Upon activation, p38 phosphorylates MK2, thereby inducing a conformational change of the autoinhibitory domain of MK2 and exposing the active site for substrate binding. Once MK2 is phosphorylated, the p38-MK2 dimer is translocated to the cytoplasm, where it forms a quaternary complex with the Hsp27-Akt dimer. See Zheng et al., The Journal of Biological Chemistry, vol. 281, no. 48, 37215-37226, Dec. 1, 2006. Hsp27 is then phosphorylated by MK2, resulting in degradation of the quaternary complex and the release of p-Hsp27 monomers and dimers. Because inhibition of MK2 blocks phosphorylation of Hsp27, without wishing to be bound by theory, it is believed that inhibition of MK2 prevents degradation of the p38-MK2-Akt-Hsp27 quaternary complex, thereby altering downstream effects. Consequent to the inhibition of quaternary complex degradation, the amount of quaternary complex would thereby increase. Moreover, the equilibrium of p38 and MK2 between the cytoplasm and nucleus would be shifted towards the cytoplasm.

Interestingly, transport of the MK2/p38 complex out of the nucleus does not require catalytically active MK2, as the active site mutant, Asp207Ala, is still transported to the cytoplasm. Phosphorylation of human MK2 by p38 on residues T222, 5272 and T334 is thought to activate the enzyme by inducing a conformational change of the autoinhibitory domain thus exposing the active site for substrate binding. Mutations of two autoinhibitory domain residues W332A and K326E in murine MK2 demonstrate an increase in basal activity and a C-terminal deletion of the autoinhibitory domain renders the enzyme constitutively active, providing additional evidence to the role of this domain in inhibition of MK2 activity.

MK2 inhibitors are useful in treating, e.g., autoimmune disorders, chronic inflammatory disorders, acute inflammatory disorders, and auto-inflammatory disorders. Accordingly, in some embodiments, the present invention provides a method of modulating a MK2-mediated inflammatory or autoimmune process.

### Compound 1 is an Irreversible MK2 Inhibitor

United States published patent application number U.S. 2016/0075720, published Mar. 17, 2016 (“the '720 publication,” the entirety of which is hereby incorporated herein by reference), describes certain compounds which covalently and irreversibly inhibit activity of MK2. Such compounds include Compound 1, which is designated as compound number 1-82 in the '720 publication, and its synthesis is described in detail at Example 82. Compound 1 is active in a variety of enzymatic and cellular assays and therapeutic models demonstrating covalent, irreversible inhibition of MK2. Notably, Compound 1 was found to inhibit phosphorylation of Hsp27, the substrate for MK2. See Example 138 of the '720 publication.

Compound 1 potently inhibits the kinase activity of MK2 in a biochemical assay with a 50% inhibitory concentration (IC₅₀) of 156.3±5.5 nM and an apparent inactivation constant (k_{inact})/apparent inhibition constant (K_{I}) (k_{inact}/K_{I}) ratio of (4.94±0.63)×103 M⁻¹s⁻¹. Compound 1 covalently modifies recombinant MK2 at Cys140. In a cellular setting, Compound 1 potently inhibits MK2 activity in human THP-1 cells with half-maximal effective concentrations (EC₅₀) for inhibition of the phosphorylation of heat shock protein 27 (pHsp27) of 89±2.6 nM and an EC₅₀ for occupancy (represented as % free MK2) of 164±18 nM.

In vivo, Compound 1 has been shown to have pharmacologic activity in animal models of human disease, including a mouse model of collagen antibody induced arthritis (CAIA), a mouse model of imiquimod-induced ear swelling, and a mouse model of mannan-induced psoriatic arthritis (PsA) disease and in an HLA-B27 transgenic model of ankylosing spondylitis in rats. In each disease model, steady-state pharmacokinetics and occupancy of MK2 were quantified. Efficacy in pharmacology models of disease was associated with MK2 target occupancy of 40% to 70%. In an ex vivo evaluation of human samples, Compound 1 significantly inhibited the production of several cytokines and chemokines produced by ankylosing spondylitis patient cells in vitro, including TNF-α, interleukin (IL)-17A, monocyte chemoattractant protein 1 (MCP-1), and IL-6. T helper 17 (Th17) cells producing the proinflammatory cytokine IL-17 have been implicated in spondyloarthritis, which has been confirmed by the efficacy of anti-IL-17 biologics in ankylosing spondylitis and PsA (Baeten, et al. Secukinumab, an interleukin-17A inhibitor, in ankylosing spondylitis. N Engl J Med. 2015; 373(26):2534-48; Mease, et al. Secukinumab: a new treatment option for psoriatic arthritis. Rheumatol Ther. 2016 June; 3(1):5-29). Accordingly, the present invention encompasses the recognition that Compound 1 is useful for treating one or more disorders associated with activity of MK2.

In some embodiments, the present invention provides methods of treating, stabilizing or lessening the severity or progression of one or more diseases or disorders associated with MK2, comprising administering to a patient in need thereof a therapeutically effective amount of Compound 1, or a pharmaceutically acceptable composition thereof.

Diseases or disorders associated with MK2 that are treated by Compound 1 include autoimmune disorders, chronic inflammatory disorders, acute inflammatory disorders, auto-inflammatory disorders, fibrotic disorders, metabolic disorders, neoplasias, or cardiovascular or cerebrovascular disorders. Thus, in some embodiments, the present invention provides a method for treating an MK2-mediated disease or disorder in a patient in need thereof, wherein said method comprises administering to said patient a therapeutically effective amount of Compound 1, or composition thereof. Such MK2-mediated diseases or disorders include, but are not limited to those described herein.

In some embodiments, the MK2-mediated disease or disorder is an autoimmune disorder, chronic and/or acute inflammatory disorder, and/or auto-inflammatory disorder. Exemplary autoimmune and/or inflammatory and/or auto-inflammatory disorders include: inflammatory bowel diseases (for example, ulcerative colitis or Crohn's disease), multiple sclerosis, psoriasis, arthritis, rheumatoid arthritis, osteoarthritis, juvenile arthritis, psoriatic arthritis, reactive arthritis, ankylosing spondylitis, cryopyrin associated periodic syndromes, Muckle-Wells syndrome, familial cold auto-inflammatory syndrome, neonatal-onset multisystem inflammatory disease, TNF receptor associated periodic syndrome, acute and chronic pancreatitis, atherosclerosis, gout, ankylosing spondylitis, fibrotic disorders (for example, hepatic fibrosis or idiopathic pulmonary fibrosis), nephropathy, sarcoidosis, scleroderma, anaphylaxis, diabetes (for example, diabetes mellitus type 1 or diabetes mellitus type 2), diabetic retinopathy, Still's disease, vasculitis, sarcoidosis, pulmonary inflammation, acute respiratory distress syndrome, wet and dry age-related macular degeneration, autoimmune hemolytic syndromes, autoimmune and inflammatory hepatitis, autoimmune neuropathy, autoimmune ovarian failure, autoimmune orchitis, autoimmune thrombocytopenia, silicone implant associated autoimmune disease, Sjogren's syndrome, familial Mediterranean fever, systemic lupus erythematosus, vasculitis syndromes (for example, temporal, Takayasu's and giant cell arteritis, Behcet's disease or Wegener's granulomatosis), vitiligo, secondary hematologic manifestation of autoimmune diseases (for example, anemias), drug-induced autoimmunity, Hashimoto's thyroiditis, hypophysitis, idiopathic thrombocytic pupura, metal-induced autoimmunity, myasthenia gravis, pemphigus, autoimmune deafness (for example, Meniere's disease), Goodpasture's syndrome, Graves' disease, HW-related autoimmune syndromes, Gullain-Barre disease, Addison's disease, anti-phospholipid syndrome, asthma, atopic dermatitis, Celiac disease, Cushing's syndrome, dermatomyositis, idiopathic adrenal adrenal atrophy, idiopathic thrombocytopenia, Kawasaki syndrome, Lambert-Eaton Syndrome, pernicious anemia, pollinosis, polyarteritis nodosa, primary biliary cirrhosis, primary sclerosing cholangitis, Raynaud's, Reiter's Syndrome, relapsing polychondritis, Schmidt's syndrome, thyrotoxidosis, sepsis, septic shock, endotoxic shock, exotoxin-induced toxic shock, gram negative sepsis, toxic shock syndrome, glomerulonephritis, peritonitis, interstitial cystitis, hyperoxia-induced inflammations, chronic obstructive pulmonary disease (COPD), vasculitis, graft vs. host reaction (for example, graft vs. host disease), allograft rejections (for example, acute allograft rejection or chronic allograft rejection), early transplantation rejection (for example, acute allograft rejection), reperfusion injury, pain (for example, acute pain, chronic pain, neuropathic pain, or fibromyalgia), chronic infections, meningitis, encephalitis, myocarditis, gingivitis, post-surgical trauma, tissue injury, traumatic brain injury, enterocolitis, sinusitis, uveitis, ocular inflammation, optic neuritis, gastric ulcers, esophagitis, peritonitis, periodontitis, dermatomyositis, gastritis, myositis, polymyalgia, pneumonia and bronchitis.

In some embodiments, the MK2-mediated disease or disorder is a fibrotic disorder. Exemplary fibrotic disorders include systemic sclerosis/scleroderma, lupus nephritis, connective tissue disease, wound healing, surgical scarring, spinal cord injury, CNS scarring, acute lung injury, pulmonary fibrosis (for example, idiopathic pulmonary fibrosis or cystic fibrosis), chronic obstructive pulmonary disease, adult respiratory distress syndrome, acute lung injury, drug-induced lung injury, glomerulonephritis, chronic kidney disease (for example, diabetic nephropathy), hypertension-induced nephropathy, alimentary track or gastrointestinal fibrosis, renal fibrosis, hepatic or biliary fibrosis, liver fibrosis (for example, nonalcoholic steatohepatitis, hepatitis C, or hepatocellular carcinoma), cirrhosis (for example, primary biliary cirrhosis or cirrhosis due to fatty liver disease (for example, alcoholic and nonalcoholic steatosis)), radiation-induced fibrosis (for example, head and neck, gastrointestinal or pulmonary), primary sclerosing cholangitis, restenosis, cardiac fibrosis (for example, endomyocardial fibrosis or atrial fibrosis), opthalmic scarring, fibrosclerosis, fibrotic cancers, fibroids, fibroma, fibroadenomas, fibrosarcomas, transplant arteriopathy, keloid, mediastinal fibrosis, myelofibrosis, retroperitoneal fibrosis, progressive massive fibrosis, and nephrogenic systemic fibrosis.

In some embodiments, the MK2-mediated disease or disorder is a metabolic disorder. Exemplary metabolic disorders include obesity, steroid-resistance, glucose intolerance, and metabolic syndrome.

In some embodiments, the MK2-mediated disease or disorder is a neoplasia. Exemplary neoplasias include cancers. In some embodiments, exemplary neoplasias include angiogenesis disorders, multiple myeloma, leukemias (for example, acute lymphocytic leukemia, acute and chronic myelogenous leukemia, chronic lymphocytic leukemia, acute lymphoblastic leukemia, or promyelocytic leukemia), lymphomas (for example, B-cell lymphoma, T-cell lymphoma, mantle cell lymphoma, hairy cell lymphoma, Burkitt's lymphoma, mast cell tumors, Hodgkin's disease or non-Hodgkin's disease), myelodysplastic syndrome, fibrosarcoma, rhabdomyosarcoma; astrocytoma, neuroblastoma, glioma and schwannomas; melanoma, seminoma, teratocarcinoma, osteosarcoma, xenoderma pigmentosum, keratoctanthoma, thyroid follicular cancer, Kaposi's sarcoma, melanoma, teratoma, rhabdomyosarcoma, metastatic and bone disorders, as well as cancer of the bone, mouth/pharynx, esophagus, larynx, stomach, intestine, colon, rectum, lung (for example, non-small cell lung cancer or small cell lung cancer), liver, pancreas, nerve, brain (for example, glioma or glioblastoma multiforme), head and neck, throat, ovary, uterus, prostate, testis, bladder, kidney, breast, gall bladder, cervix, thyroid, prostate, and skin.

In some embodiments, the MK2-mediated disorder is a cardiovascular or cerebrovascular disorder. Exemplary cardiovascular disorders include atherosclerosis, restenosis of an atherosclerotic coronary artery, acute coronary syndrome, myocardial infarction, cardiac-allograft vasculopathy and stroke. Exemplary cerebrovascular diseases include central nervous system disorders with an inflammatory or apoptotic component, Alzheimer's disease, Parkinson's disease, Huntington's disease, amyotrophic lateral sclerosis, spinal cord injury, neuronal ischemia and peripheral neuropathy.

In some embodiments, the disease or disorder associated with MK2 is an autoimmune disease or disorder. In some embodiments, the disease or disorder associated with MK2 is an inflammatory disease or disorder. In some such embodiments, the inflammatory disease or disorder is selected from a chronic inflammatory disorder, an acute inflammatory disorder, or an auto-inflammatory disorder. In some embodiments, such autoimmune or inflammatory diseases and disorders are selected from rheumatoid arthritis, psoriatic arthritis, psoriasis, and ankylosing spondylitis.

In some embodiments, the present invention provides a method of preventing the progression of an autoimmune or inflammatory disease or disorder associated with MK2, comprising administering to a patient in need thereof a therapeutically effective amount of Compound 1, or a pharmaceutically acceptable composition thereof. In some embodiments, such autoimmune or inflammatory diseases and disorders are selected from rheumatoid arthritis, psoriatic arthritis, psoriasis, and ankylosing spondylitis.

Ankylosing Spondylitis

Ankylosing spondylitis (AS) is a chronic form of arthritis that primarily affects the spine, although other joints can become involved. A systemic inflammatory disease of indeterminate etiology, ankylosing spondylitis affects the axial spine (spondylitis), with sacroiliitis as its hallmark. The most common presenting symptom is chronic back pain and progressive spinal stiffness, a result of inflammation affecting the spine and sacroiliac joints (Feld et al. Axial disease in psoriatic arthritis and ankylosing spondylitis: a critical comparison. Nat Rev Rheumatol 2018; 14(6):363-71). In more advanced cases this inflammation can lead to ankylosis—new bone formation in the spine—causing sections of the spine to fuse in a fixed, immobile position.

Ankylosing spondylitis can also cause inflammation, pain, and stiffness in other areas of the body such as the shoulders, hips, ribs, heels, and small joints of the hands and feet. Sometimes the eyes can become involved (known as iritis or uveitis), and—rarely—the lungs and heart can be affected. The hallmark feature of ankylosing spondylitis is the involvement of the sacroiliac (SI) joints during the progression of the disease. The SI joints are located at the base of the spine, where the spine joins the pelvis.

Ankylosing spondylitis is typically diagnosed in people younger than 40 years and about 80% of patients develop first symptoms when they are younger than 30 years (Hanson et al. Genetics and the Causes of Ankylosing Spondylitis. Rheum Dis Clin North Am. 2017; 43(3):401-14). It is estimated that approximately 70% of patients with AS are males (de Winter et al. Prevalence of peripheral and extra-articular disease in ankylosing spondylitis versus non-radiographic axial spondyloarthritis: a meta-analysis. Arthritis Res Ther 2016; 18:196) Recent studies reported the prevalence of AS to range from 9 to 30 per 10,000 in the general population, depending on geographic area, study population or data source, case definition, and ascertainment methods. In general, there is a clear correlation between the prevalence of AS in a given population and the prevalence of HLA-B27 in that group, with the prevalence of AS being approximately 5 to 6 percent among people who are HLA-B27-positive (Reveille et al. The Epidemiology of Back Pain, Axial Spondyloarthritis and HLA-B27 in the United States. Am J Med Sci. 2013; 345(6): 431-6). Approximately 94% of individuals with AS are HLA-B27-positive (Brown et al. HLA class I associations of ankylosing spondylitis in the white population in the United Kingdom. Ann Rheum Dis. 1996; 55(4):268-70).

Although HLA-B27 is the largest single genetic contributor to disease pathophysiology, many other genetic loci, including those associated with the interleukin (IL)-17A pathway, have been associated with AS (Brown et al. Genetics of ankylosing spondylitis—insights into pathogenesis. Nat Rev Rheumatol. 2016; 12(2):81-91; Costantino et al. Genetics and Functional Genomics of Spondyloarthritis. Front Immunol. 2018:9:2933). Chronic inflammation in AS is thought to be driven by CD4+ and/or CD8+ T lymphocytes, including innate-like lymphocytes, and cytokines such as tumor necrosis factor (TNF)-α and IL-17A (Ranganathan et al. Macrophage Migration Inhibitory Factor Induces Inflammation and Predicts Spinal Progression in Ankylosing Spondylitis. Arthritis Rheumatol 2017; 69(9):1796-1806). Classification criteria for AS were proposed based on clinical grounds in the 1960s and later modified to include radiological criteria, known as the modified New York criteria for diagnosis of AS (van der Linden et al. Evaluation of Diagnostic Criteria for Ankylosing Spondylitis. A Proposal for Modification of the New York Criteria. Arthritis Rheum. 1984; 27(4):361-8). More recently, the Assessment of SpondyloArthritis international Society (ASAS) formulated classification criteria for axial spondyloarthritis (axSpA), of which AS is considered the prototype disease, based on imaging, clinical, and laboratory criteria (Rudwaleit et al. The development of Assessment of SpondyloArthritis international Society classification criteria for axial spondyloarthritis (part I): classification of paper patients by expert opinion including uncertainty appraisal. Ann Rheum Dis. 2009; 68(6):770-6; Rudwaleit et al. The development of Assessment of SpondyloArthritis international Society classification criteria for axial spondyloarthritis (part II): validation and final selection. Ann Rheum Dis 2009; 68(6):777-83). Disease classification of axSpA is established in persons with a history of back pain for 3 or more consecutive months before reaching 45 years of age, the presence of sacroiliitis confirmed on magnetic resonance imaging (MM) or plain radiography, and with at least one clinical or laboratory finding that is characteristic of spondyloarthritis (SpA). Alternatively, persons with this history who have a positive test result for HLA-B27 and ≥2 clinical or laboratory features of SpA also fulfill the classification criteria for axSpA. Individuals with axSpA who have established radiographic evidence of sacroiliitis are considered to have met the definition for AS (Rudwaleit et al. The development of Assessment of SpondyloArthritis international Society classification criteria for axial spondyloarthritis (part I): classification of paper patients by expert opinion including uncertainty appraisal. Ann Rheum Dis. 2009; 68(6):770-6; Rudwaleit et al. The development of Assessment of SpondyloArthritis international Society classification criteria for axial spondyloarthritis (part II): validation and final selection. Ann Rheum Dis 2009; 68(6):777-83).

The treatment goal in patients with AS is to optimize long-term health-related quality of life and social participation through control of signs and symptoms, prevention of structural damage, normalization or preservation of function, avoidance of toxicities and minimization of comorbidities (Smolen et al. Treating axial spondyloarthritis and peripheral spondyloarthritis, especially psoriatic arthritis, to target: 2017 update of recommendations by an international task force. Ann Rheum Dis. 2018; 77(1):3-17). Current treatment guidelines for active AS (Bath Ankylosing Spondylitis Disease Activity Index [BASDAI] of at least 4, or Ankylosing Spondylitis Disease Activity Score-C-reactive protein [ASDAS-CRP] of at least 2.1) strongly recommend the use of nonsteroidal anti-inflammatory drugs (NSAIDs) and conditionally recommend their continuous use, based on very low-quality evidence (van der Heijde D, et al. 2016 update of the ASAS-EULAR management recommendations for axial spondyloarthritis. Ann Rheum Dis. 2017; 76(6):978-91). Tumor necrosis factor (TNF) blockers and anti-IL-17A monoclonal antibody (mAb) agents have become standard of care for patients who are unresponsive or intolerant to NSAIDs. Based on results of pivotal trials of currently approved biologics in AS, about 30% to 40% of patients treated with biologics do not achieve an Assessment of SpondyloArthritis International Society Response Criteria with an improvement of at least 20% (ASAS 20) and up to 64% of patients do not achieve an Assessment of SpondyloArthritis International Society Response Criteria with an improvement of at least 40% (ASAS 40) (Sieper et al. Secukinumab efficacy in anti-TNF-naive and anti-TNF-experienced subjects with active ankylosing spondylitis: results from the MEASURE 2 Study. Ann Rheum Dis. 2017; 76:571-75; Deodhar et al. Efficacy and Safety of Ixekizumab in the Treatment of Radiographic Axial Spondyloarthritis: Sixteen-Week Results From a Phase III Randomized, Double-Blind, Placebo-Controlled Trial in Patients With Prior Inadequate Response to or Intolerance of Tumor Necrosis Factor Inhibitors. Arthritis Rheumatol 2019; 71(4):599-611).

Although biologics can reduce inflammation and improve symptoms, there is only indirect evidence that currently available biologic TNF blockers influence spinal radiographic progression (Haroon et al. Effect of TNF-alpha inhibitor treatment on bone mineral density in patients with ankylosing spondylitis: A systematic review and meta-analysis. Semin Arthritis Rheum. 2014; 44(22):155-61; Maas et al. Reduction in Spinal Radiographic Progression in Ankylosing Spondylitis Patients Receiving Prolonged Treatment With Tumor Necrosis Factor Inhibitors. Arthritis Care Res (Hoboken). 2017; 69(7):1011-19; Molnar et al. TNF blockers inhibit spinal radiographic progression in ankylosing spondylitis by reducing disease activity: results from the Swiss Clinical Quality Management cohort. Ann Rheum Dis. 2018; 77(1):63-69), which continues to occur in spite of treatment (Poddubnyy et al. Physical Function and Spinal Mobility Remain Stable Despite Radiographic Spinal Progression in Patients with Ankylosing Spondylitis Treated with TNF-α Inhibitors for Up to 10 Years. J Rheumatol 2016; 43(12); 2142-8). Biologics require parenteral administration and are associated with development of autoantibodies, which may be neutralizing and limit drug effectiveness. In addition, profound TNF inhibition by currently available TNF-directed biologics is associated with increased risks of serious infections and malignancies.

In some embodiments, the present disclosure provides the recognition that AS patients who fail or cannot tolerate NSAIDs, and those who have also failed therapy with biologic agents, represent a patient population with high unmet medical need for whom there are currently no approved oral medications available to treat the underlying disease.

In some embodiments, the present invention provides a method for treating or lessening the severity of ankylosing spondylitis in a patient, comprising administering to the patient Compound 1. In some embodiments, Compound 1 is administered to a subject who has radiologically confirmed AS. In some such embodiments, the subject has had an inadequate response to nonsteroidal anti-inflammatory drugs (NSAIDs).

In some embodiments, the term “treating or lessening the severity of ankylosing spondylitis” refers to the improvement of long-term health-related quality of life and social participation through one or more of (i) control of signs and symptoms of AS, (ii) prevention of structural damage, (iii) normalization or preservation of function, and (iv) avoidance of toxicities and minimization of comorbidities.

In some embodiments, the present disclosure provides a method of administering Compound 1 to a subject who is HLA-B-27-positive. In some embodiments, provided methods comprise administering Compound 1 to a subject in need thereof, wherein the subject is suffering from chronic inflammation associated with or mediated by one or more lymphocytes and/or cytokines. In some such embodiments, the one or more lymphocytes and/or cytokines is or are selected from CD4+ T lymphocytes, CD8+ T lymphocytes, innate-like lymphocytes, tumor necrosis factor (TNF)-α, and IL-17A.

In some embodiments, the present disclosure provides a method of administering Compound 1 to a subject who satisfies the classification criteria for axial spondyloarthritis (axSpA). In some such embodiments, the classification criteria for axSpA is based on imaging, clinical, and laboratory criteria. In some embodiments, a subject has or is diagnosed with radiographic axSpA. In some embodiments, a subject has or is diagnosed with non-radiographic axSpA. Such subjects exhibit clinical signs and symptoms of SpA but does not exhibit characteristic radiographic changes on pelvic X-rays.

In some embodiments, a subject who satisfies the classification criteria for axSpA is a subject who has a history of back pain for 3 or more consecutive months before reaching 45 years of age, confirmed sacroiliitis, and at least one clinical or laboratory finding that is characteristic of spondyloarthritis (SpA). As used herein, “confirmed sacroiliitis” means sacroiliitis that is or has been confirmed on magnetic resonance imaging (MM) or plain radiography. In some embodiments, a subject who satisfies the classification criteria for axSpA is a subject who has a positive test result for HLA-B27 and ≥2 clinical or laboratory features of SpA. In some embodiments, a subject suffering from AS is a subject who has axSpa and has established radiographic evidence of sacroiliitis.

In some embodiments, the present disclosure provides a method of preventing or slowing the progression of structural damage and/or preservation of function in a subject who is suffering from or has been diagnosed with ankylosing spondylitis. In some embodiments, a subject suffering from or diagnosed with ankylosing spondylitis exhibits one or more of the following criteria:
a. low back pain and stiffness for more than 3 months that improves with exercise, but is not relieved by rest;
b. limitation of motion of the lumbar spine in the sagittal and frontal planes;
c. limitation of chest expansion relative to normal values correlated for age and sex; and
d. sacroiliitis grade ≥2 bilaterally or grade 3 to 4 unilaterally.

In some embodiments, a subject has been diagnosed with AS according to the Modified New York Criteria for Ankylosing Spondylitis (1984). In some embodiments, a subject has symptoms of active AS based on a Bath Ankylosing Spondylitis Disease Activity Index (BASDAI) score ≥4. In some embodiments, a subject has a total Back Pain Numerical Rating Scales (NRS) score ≥4. In some embodiments, a subject meets one or more of the following criteria:
a. diagnosed with AS according to the Modified New York Criteria for Ankylosing Spondylitis (1984);
b. symptoms of active AS based on a BASDAI score ≥4; and
c. a total Back Pain Numerical Rating Scales (NRS) score ≥4.

In some embodiments, the present invention provides a method of treating AS in a subject, the method comprising:

administering to the subject Compound 1,
wherein the subject experiences improvement or response in at least three of the following Assessment in SpondyloArthritis International Society (ASAS) criteria:
   a. patient global assessment of disease;
   b. total back pain;
   c. function; and
   d. inflammation.

In some embodiments, the subject experiences improvement or response in at least three of the ASAS criteria of at least 20% and a minimum of one unit on a scale of 0 to 10 and, for the remaining criterion, the subject experiences no worsening from baseline of no more than 20% and a minimum of one unit on a scale of 0 to 10. In some such embodiments, such improvement or response criteria are known as the “ASAS 20 improvement criteria.”

Accordingly, in some embodiments, the present invention provides a method of treating AS in a subject, the method comprising:

administering to the subject Compound 1,
wherein the subject experiences improvement or response of at least 20% and a minimum of one unit in at least three of the following ASAS criteria:
   a. patient global assessment of disease (0 to 10 numerical rating scale);
   b. total back pain (0 to 10 numerical rating scale);
   c. function (assessed by Bath Ankylosing Spondylitis Functional Index (BASFI)); and
   d. inflammation (mean of numerical rating scales for Questions #5 and #6 on Bath Ankylosing Spondylitis Disease Activity Index (BASDAI)); and
wherein, for the remaining criterion, the subject experiences no worsening from baseline of greater than 20% and a minimum of one unit on a scale of 0 to 10.

In some embodiments, the present disclosure provides a method of improving disease activity (e.g., signs and symptoms of AS) in a subject who is suffering from or has been diagnosed with AS, the method comprising administering to the subject Compound 1, wherein disease activity is assessed by the ASAS 20 improvement criteria.

In some embodiments, the subject experiences improvement or response in at least three of the ASAS criteria of at least 40% and a minimum of two units on a scale of 0 to 10 and, for the remaining criterion, the subject experiences no worsening from baseline. In some embodiments, the subject experiences improvement or response in at least three of the ASAS criteria of at least 40% and a minimum of two units on a scale of 0 to 10 and, for the remaining criterion, the subject experiences no worsening from baseline of no more than 20% and a minimum of one unit on a scale of 0 to 10. In some such embodiments, such improvement or response criteria are known as the “ASAS 40 improvement criteria.”

Accordingly, in some embodiments, the present invention provides a method of treating AS in a subject, the method comprising:

administering to the subject Compound 1,
wherein the subject experiences improvement or response of at least 40% and a minimum of two units in at least three of the following ASAS criteria:
   a. patient global assessment of disease (0 to 10 numerical rating scale);
   b. total back pain (0 to 10 numerical rating scale);
   c. function (assessed by Bath Ankylosing Spondylitis Functional Index (BASFI)); and
   d. inflammation (mean of numerical rating scales for Questions #5 and #6 on Bath Ankylosing Spondylitis Disease Activity Index (BASDAI)); and
wherein, for the remaining criterion, the subject experiences no worsening from baseline of greater than 20% and a minimum of one unit on a scale of 0 to 10.

In some embodiments, the present disclosure provides a method of improving disease activity (e.g., signs and symptoms of AS) in a subject who is suffering from or has been diagnosed with AS, the method comprising administering to the subject Compound 1, wherein disease activity is assessed by the ASAS 40 improvement criteria.

In some embodiments, the present disclosure provides a method of improving disease activity (e.g., signs and symptoms of AS) in a subject who is suffering from or has been diagnosed with AS, the method comprising administering to the subject Compound 1, wherein disease activity is assessed by the Ankylosing Spondylitis Disease Activity Score—C-reactive protein (ASDAS-CRP). In some embodiments, the subject achieves a ASDAS-CRP score of ≥1.1. In some such embodiments, the subject achieves a ASDAS-CRP score of ≥2.0. In some such embodiments, the subject achieves a ASDAS-CRP score of <1.3.

In some embodiments, the present disclosure provides a method of improving disease activity (e.g., signs and symptoms of AS) in a subject who is suffering from or has been diagnosed with AS, the method comprising administering to the subject Compound 1, wherein disease activity is assessed by the Bath Ankylosing Spondylitis Disease Activity Index (BASDAI).

In some embodiments, the present disclosure provides a method of improving physical function in a subject who is suffering from or has been diagnosed with AS, the method comprising administering to the subject Compound 1, wherein physical function is assessed by the Bath Ankylosing Spondylitis Functional Index (BASFI).

In some embodiments, the present disclosure provides a method of reducing spinal and sacroiliac joint inflammation in a subject who is suffering from or has been diagnosed with AS, the method comprising administering to the subject Compound 1, wherein spinal and sacroiliac joint inflammation is assessed by Spondylarthritis Research Consortium of Canada (SPARCC) MM score of sacroiliac joints and spine.

In some embodiments, a subject suffering from or diagnosed with ankylosing spondylitis has failed therapy with at least 2 nonsteroidal anti-inflammatory drugs (NSAIDs). In some embodiments, a subject suffering from or diagnosed with ankylosing spondylitis has not received therapy selected from one or more of:
a. a cell depleting biologic agent such as an anti-CD20 antibody (e.g., rituximab), an anti-CD4 antibody, an anti-CD3 antibody, denosumab, an anti-IL-6 antibody (e.g., tocilizumab and sarilumab), and an anti-IL-23 antibody (e.g., ustekinuma) for at least 6 months prior to administration of Compound 1;
b. an oral corticosteroid (e.g., prednisone, etc.) in an amount greater than 10 mg/day systemically for at least 2 weeks prior to administration of Compound 1;
c. an intramuscular, intravenous, or intraarticular corticosteroid in any amount within at least 4 weeks of administration of Compound 1;
d. a vitamin K antagonist (e.g., warfarin);
e. isoniazid within at least 4 weeks of administration of Compound 1; and
f. any medication that is a substrate of one or more of the following transporters and has a narrow therapeutic index: p-glycoprotein (P-gp) (e.g., aliskiren, ambrisentan, colchicine, cyclosporine, dabigatran etexilate, digoxin, everolimus, fexofenadine, methotrexate, ranolazine, rivaroxaban, saxagliptin, sirolimus, sitagliptin, talinolol, ticagrelor, tolvaptan, etc.), breast cancer resistance protein (BCRP) (e.g., methotrexate, sulfasalazine, leflunomide, rosuvastatin, etc.), organic cation transporter 1 (OCT1) (e.g., metformin, gabapentin, pramipexole, tramadol, varenicline, etc.), organic anion transporting polypeptides 1B1 and 1B3 (OATP1B1 and OATP1B3, respectively) (e.g., ambrisentan, atorvastatin, ezetimibe, fluvastatin, glyburide, rosuvastatin, simvastatin acid, pitavastatin, pravastatin, repaglinide, telmisartan, valsartan, olmesartan, mycophenolic acid, etc.).

In some embodiments, a subject suffering from or diagnosed with ankylosing spondylitis has failed therapy with at least 2 nonsteroidal anti-inflammatory drugs (NSAIDs) and not more than 1 biological agent. In some embodiments, a subject who has failed therapy with not more than 1 biological agent is a subject who has had, for at least 12 weeks, an inadequate response and/or an unacceptable safety/tolerability to at least 1 dose of a biologic agent for AS (e.g., a TNF antagonist or IL-17A monoclonal antibody).

In some such embodiments, the patient is administered a therapeutically effective amount of Compound 1. In some embodiments, the patient is administered a unit dose of Compound 1.

In some embodiments, the present invention provides a use of Compound 1 in the manufacture of a medicament for treating ankylosing spondylitis. In some embodiments, the present invention provides Compound 1 for use in treating ankylosing spondylitis.

Biomarkers of AS. In some embodiments, the present disclosure provides a method of administering to a subject Compound 1 and monitoring the level of one or more biomarkers associated or correlated with AS. Compound 1 is a potent and selective inhibitor of MK2 in biochemical and cellular assays; moreover, it is an effective inhibitor of inflammation in vivo in animal models and of pro-inflammatory cytokines in vitro in patient-derived cells. These pro-inflammatory cytokines and chemokines include TNF-α, monocyte chemoattractant protein-1 (MCP-1), and IL-17A and have been shown to be increased in AS patients (Braun et al. Anti-tumour necrosis factor a therapy for ankylosing spondylitis: international experience. Ann Rheum Dis 2002; 61(Suppl III): iii51-iii60; West et al. Oncostatin M drives intestinal inflammation and predicts response to tumor necrosis factor-neutralizing therapy in patients with inflammatory bowel disease. Nat Med. 2017; 23 (5)579-89; Romero-Sanchez et al. Serum monocyte chemotactic protein-1 concentrations distinguish patients with ankylosing spondylitis from patients with mechanical low back pain. J Spinal Disord Tech. 2011; 24(3):202-7). Additionally, increased serum levels of TNF-α have been correlated with increased CRP levels in AS patients (Wagner et al. Serum markers associated with clinical improvement in patients with ankylosing spondylitis treated with golimumab. Ann Rheum Dis. 2012; 71(5):674-80). Accordingly, in some embodiments, a biomarker associated or correlated with AS is selected from a pro-inflammatory cytokine or chemokine. In some such embodiments, a pro-inflammatory cytokine or chemokine is selected from TNF-α, monocyte chemoattractant protein-1 (MCP-1), and IL-17A. In some embodiments, the level of a pro-inflammatory cytokine or chemokine decreases over a period of time relative to a reference standard. In some such embodiments, a reference standard is the level of the pro-inflammatory cytokine or chemokine for a given subject or a given population prior to exposure to Compound 1.

In AS, several bone remodeling processes take place simultaneously: pathologic new bone formation in the form of syndesmophytes and bone loss in the form of bone erosion, osteolysis, and bone mineral density (BMD) loss leading to osteoporosis (Klingberg et al. Osteoporosis in ankylosing spondylitis—prevalence, risk factors and methods of assessment. Arthritis Res Ther 2012:14 (3):R108). Compound 1 has been shown to reduce bone resorption activity in vitro. Accordingly, in some embodiments, a biomarker associated or correlated with AS is a bone formation marker. In some such embodiments, a bone formation marker is selected from procollagen type 1 N-terminal propeptide (P1NP) and bone resorption markers such as carboxy terminal cross-linked telopeptide of type 1 collagen (CTX-1). In some embodiments, the level of a bone formation marker decreases over a period of time relative to a reference standard. In some embodiments, the level of a bone formation marker increases over a period of time relative to a reference standard. In some embodiments, a reference standard is the level of the bone formation marker for a given subject or a given population prior to exposure to Compound 1.

Bone destruction is mediated by the recruitment of osteoclast precursors (OCPs) into the inflamed tissue and their differentiation into mature osteoclasts. TNF inhibition has resulted in sustained loss of circulating OCPs that can differentiate into osteoclasts (Lam et al. TNF-alpha induces osteoclastogenesis by direct stimulation of macrophages exposed to permissive levels of RANK ligand. J Clin Invest. 2000; 106(12):1481-8; Li et al. Systemic tumor necrosis factor alpha mediates an increase in peripheral CD11bhigh osteoclast precursors in tumor necrosis factor alpha-transgenic mice. Arthritis Rheum. 2004; 50(1):265-76). Compound 1 was found to inhibit osteoclast differentiation in vitro, suggesting that MK2 inhibition may result in a similar reduction in osteoclast precursors in AS subjects. Accordingly, in some embodiments, the biomarker associated or correlated with AS is an osteoclast precursor (OCP). In some embodiments, the level of an osteoclast precursor decreases over a period of time relative to a reference standard. In some such embodiments, a reference standard is the level of the osteoclast precursor for a given subject or a given population prior to exposure to Compound 1.

In some embodiments, a biomarker associated or correlated with AS is a genetic marker. In some such embodiments, a genetic marker is selected from HLA-B27 and polygenic risk scores built using public AS data (see, e.g., Rostami et al. Prediction of Ankylosing Spondylitis in the HUNT Study by a Genetic Risk Score Combining 110 Single-nucleotide Polymorphisms of Genome-wide Significance. J Rheumatol 2019; 46:1-7).

Rheumatoid Arthritis

Rheumatoid arthritis is a chronic autoimmune disorder in which the body's immune system attacks its own tissue, including joint linings, synovial tissues, cartilage and bone, causing painful swelling. The inflammation that results from immune system attacks results in the thickening of the synovium, the tissue that lines the insides of joints, leading to swelling and pain in and around the joints. Over long periods of time, the inflammation associated with rheumatoid arthritis can damage cartilage, the elastic tissue that covers the ends of bones in a joint, as well as the bones themselves. Over time, there is loss of cartilage, and the joint spacing between bones can become smaller. Joints can become loose, unstable, painful and lose their mobility. Joint deformity also can occur. Joint damage cannot be reversed, and because it can occur early, doctors recommend early diagnosis and aggressive treatment to control rheumatoid arthritis. In severe cases, rheumatoid arthritis attacks internal organs.

Patients with rheumatoid arthritis can be classified into distinct subsets, including lymphoid, myeloid and fibroid subsets. Dennis et al., “Synovial phenotypes in rheumatoid arthritis correlate with response to biologic therapeutics,” Arthritis Research & Therapy 2014, 16:R90, 1-18; Setiadi, et. al, “Synovial Subset-Derived Baseline Serum Biomarkers Segregate Rheumatoid Arthritis Patients into Subgroups with Distinct Serum Protein and Clinical Characteristics,” Abstract Number 1307, 2013 ACR/ARHP Annual Meeting.

In some embodiments, the present invention provides a method of treating rheumatoid arthritis in a patient, comprising administering to the patient Compound 1. In some such embodiments, the patient is administered a therapeutically effective amount of Compound 1. In some embodiments, the patient is administered a unit dose of Compound 1.

In some embodiments, the present invention provides a method of treating one or more of the lymphoid, myeloid and fibroid subsets of rheumatoid arthritis, comprising administering Compound 1 to a patient in one or more subsets. Such subsets are classified by the presence of certain biomarkers which are detailed in Dennis et al., “Synovial phenotypes in rheumatoid arthritis correlate with response to biologic therapeutics,” Arthritis Research & Therapy 2014, 16:R90, 1-18; Setiadi, et. al, “Synovial Subset-Derived Baseline Serum Biomarkers Segregate Rheumatoid Arthritis Patients into Subgroups with Distinct Serum Protein and Clinical Characteristics,” Abstract Number 1307, 2013 ACR/ARHP Annual Meeting, each of which is hereby incorporated by reference.

In some embodiments, the present invention provides a method for treating or lessening the severity of rheumatoid arthritis in a patient, wherein the patient has one or more biomarkers for the lymphoid subset of rheumatoid arthritis, comprising administering to the patient Compound 1. Such biomarkers for the lymphoid subset of rheumatoid arthritis include, for example, high CXCL13 and low soluble ICAM1 expression levels. In some embodiments, the present invention provides a method for treating or lessening the severity of rheumatoid arthritis in a patient, wherein the patient has one or more biomarkers for the myeloid subset of rheumatoid arthritis, comprising administering to the patient Compound 1. In some embodiments, the present invention provides a method for treating or lessening the severity of rheumatoid arthritis in a patient, wherein the patient has one or more biomarkers for the fibroid subset of rheumatoid arthritis, comprising administering to the patient Compound 1. In some embodiments, the present invention provides a method for treating or lessening the severity of at least one subset of rheumatoid arthritis, comprising administering to the patient Compound 1. In some embodiments, the subset of rheumatoid arthritis is lymphoid. In some embodiments, the subset of rheumatoid arthritis is myeloid. In some embodiments, the subset of rheumatoid arthritis is fibroid.

In some embodiments, the present invention provides a use of Compound 1 in the manufacture of a medicament for treating rheumatoid arthritis. In some embodiments, the present invention provides Compound 1 for use in treating rheumatoid arthritis.

Psoriasis and Psoriatic Arthritis

Psoriasis is a chronic, inflammatory disease of the skin, scalp, nails, and joints that is characterized by a scaly rash that occurs most frequently on the elbows, knees, and scalp, but can cover much of the body. A normal skin cell matures and falls off the body's surface in 28 to 30 days, but a psoriatic skin cell takes only three to four days to mature and gathers at the surface, thus forming lesions.

Up to 30 percent of people with psoriasis also develop psoriatic arthritis. In most cases (though not always), the psoriasis will precede the arthritis, sometimes by many years. When arthritis symptoms occur with psoriasis, it is called psoriatic arthritis (PsA). In these cases, the joints at the end of the fingers are most commonly affected, causing inflammation and pain, but other joints like the wrists, knees, and ankles can also become involved. Symptoms in the fingernails and toenails range from small pits in the nails to nearly complete destruction and crumbling as seen in reactive arthritis or fungal infections.

About 20 percent of patients with PsA will develop spinal involvement, which is called psoriatic spondylitis. Inflammation of the spine can lead to complete fusion, as in ankylosing spondylitis (AS), or affect only certain areas such as the lower back or neck. Patients who are HLA-B27 positive are more likely than others to have their disease progress to the spine.

PsA and AS are considered genetically and clinically related because both are inflammatory rheumatic diseases linked to the HLA-B27 gene. HLA-B27 is a powerful predisposing gene associated with several rheumatic diseases. The gene itself does not cause disease, but can make people more susceptible. While a number of genes are linked to PsA, the highest predictive value is noted with HLA-B27.

In some embodiments, the present invention provides a method for treating or lessening the severity of psoriasis and/or psoriatic arthritis in a patient, comprising administering to the patient Compound 1. In some such embodiments, the patient is administered a therapeutically effective amount of Compound 1. In some embodiments, the patient is administered a unit dose of Compound 1.

In some embodiments, the present invention provides a use of Compound 1 in the manufacture of a medicament for treating psoriasis and/or psoriatic arthritis. In some embodiments, the present invention provides Compound 1 for use in treating psoriasis and/or psoriatic arthritis.

In some embodiments, provided methods comprise administering Compound 1 in an amount of about 3 mg to about 1000 mg. In some embodiments, Compound 1 is administered in an amount of about 3 mg to about 15 mg, about 10 mg to about 25 mg, about 15 mg to about 50 mg, about 25 mg to about 75 mg, about 50 mg to about 100 mg, about 75 mg to about 125 mg, about 100 mg to about 150 mg, or about 125 mg to about 200 mg.

In some embodiments, provided methods comprise administering Compound 1 in an amount of about 200 mg to about 300 mg, about 250 mg to about 500 mg, about 500 mg to about 750 mg, or about 750 mg to about 1000 mg.

In some embodiments, the present invention provides a method of treating a disease or disorder associated with MK2, wherein the method comprises administering to a patient in need thereof about 0.1 mg to about 10,000 mg of Compound 1. In some embodiments, provided methods comprise administering Compound 1 in an amount of about 0.1 mg to about 9000 mg, about 0.1 mg to about 8000 mg, about 0.1 to about 7000 mg, about 0.1 mg to about 6000 mg, about 0.1 mg to about 5000 mg, about 0.1 mg to about 4000 mg, about 0.1 mg to about 3000 mg, about 0.1 mg to about 2000, about 0.1 mg to about 1000 mg, about 0.1 mg to about 900 mg, about 0.1 mg to about 800 mg, about 0.1 mg to about 700 mg, about 0.1 mg to about 600 mg, about 0.1 mg to about 500 mg, about 0.1 mg to about 400 mg, about 0.1 mg to about 300 mg, about 0.1 mg to about 200 mg, about 0.1 mg to about 100 mg, about 0.1 mg to about 75 mg, about 0.1 mg to about 50 mg, about 0.1 mg to about 25 mg, about 0.1 mg to about 15 mg, about 0.1 mg to about 10 mg, about 0.1 mg to about 5 mg, about 0.1 mg to about 3 mg, or about 0.1 mg to about 1 mg.

In some embodiments, provided methods comprise administering Compound 1 in an amount of about 0.5 mg to about 500 mg, about 1 mg to about 400 mg, about 3 mg to about 300 mg, about 5 mg to about 200 mg, about 10 mg to about 150 mg, about 15 to about 100 mg, or about 25 mg to about 75 mg.

In some embodiments, provided methods comprise administering Compound 1 in an amount of about 1 mg to about 500 mg, about 3 mg to about 400 mg, about 5 mg to about 300 mg, about 10 mg to about 200 mg, about 15 mg to about 150 mg, about 25 mg to about 100 mg.

In some embodiments, provided methods comprise administering Compound 1 in an amount of about 3 mg to about 500 mg, about 5 mg to about 400 mg, about 10 mg to about 300 mg, about 15 mg to about 200 mg, about 25 mg to about 150 mg, about 50 mg to about 100 mg, or about 75 mg to about 125 mg.

In some embodiments, provided methods comprise administering Compound 1 in an amount of about 5 mg to about 500 mg, about 10 mg to about 400 mg, about 15 mg to about 300 mg, about 25 mg to about 200 mg, about 50 mg to about 150 mg, or about 75 mg to about 100 mg.

In some embodiments, provided methods comprise administering Compound 1 in an amount of about 10 mg to about 500 mg, about 15 mg to about 400 mg, about 25 mg to about 300 mg, about 50 mg to about 200 mg, about 75 mg to about 150 mg, or about 100 mg to about 125 mg.

In some embodiments, provided methods comprise administering Compound 1 in an amount of about 15 mg to about 500 mg, about 25 mg to about 400 mg, about 50 mg to about 300 mg, about 75 mg to about 200 mg, or about 100 to about 150 mg.

In some embodiments, provided methods comprise administering Compound 1 in an amount of about 25 mg to about 500 mg, about 50 mg to about 400 mg, about 75 mg to about 300 mg, about 100 to about 200 mg, or about 125 mg to about 150 mg.

In some embodiments, provided methods comprise administering Compound 1 in an amount of about 50 mg to about 500 mg, about 75 mg to about 400 mg, about 100 mg to about 300 mg, or about 150 mg to about 200 mg.

In some embodiments, provided methods comprise administering Compound 1 in an amount of about 75 mg to about 500 mg, about 100 mg to about 400 mg, or about 125 mg to about 300 mg.

In some embodiments, provided methods comprise administering Compound 1 in an amount of about 100 mg to about 500 mg, about 150 mg to about 400 mg, or about 200 mg to about 300 mg.

In some embodiments, provided methods comprise administering Compound 1 in an amount of about 200 mg to about 1000 mg, about 250 mg to about 900 mg, about 300 mg to about 800 mg, about 350 mg to about 750 mg, about 400 mg to about 700 mg, or about 500 mg to about 600 mg.

In some embodiments, the present invention provides a method of treating a disease or disorder associated with MK2, wherein the method comprises administering to a patient in need thereof about 3 mg to about 400 mg of Compound 1. In some embodiments, the present invention provides a method of treating a disease or disorder associated with MK2, wherein the method comprises administering to a patient in need thereof about 3 mg, about 10 mg, about 30 mg, about 100 mg, about 200 mg or about 400 mg of Compound 1.

In some embodiments, provided methods comprise administering Compound 1 in an amount of about 50 mg to about 150 mg. In some embodiments, provided methods comprise administering Compound 1 in an amount of about 60 mg to about 150 mg. In some embodiments, provided methods comprise administering a total daily dose of Compound 1 in an amount of 54 mg to 66 mg. In some embodiments, provided methods comprise administering a total daily dose of Compound 1 in an amount of 56 mg to 64 mg. In some embodiments, provided methods comprise administering a total daily dose of Compound 1 in an amount of 58 mg to 62 mg. In some embodiments, provided methods comprise administering a total daily dose of Compound 1 in an amount of 135 mg to 165 mg. In some embodiments, provided methods comprise administering a total daily dose of Compound 1 in an amount of 140 mg to 160 mg. In some embodiments, provided methods comprise administering a total daily dose of Compound 1 in an amount of 145 mg to 155 mg. In some embodiments, provided methods comprise administering a total daily dose of Compound 1 in an amount of 148 mg to 152 mg.

In some embodiments, provided methods comprise administering Compound 1 in an amount of about 0.1 mg, about 0.2 mg, about 0.3 mg, about 0.4 mg, about 0.5 mg, about 1 mg, about 3 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, or about 1000 mg.

In some embodiments, provided methods comprise administering Compound 1 in an amount of about 60 mg. In some embodiments, provided methods comprise administering Compound 1 in an amount of about 150 mg.

In some embodiments, provided methods comprise administering a total daily dose of Compound 1 in an amount of about 1 mg to about 5 mg, about 8 mg to about 12 mg, about 28 mg to about 32 mg, about 98 mg to about 102 mg, about 198 mg to about 202 mg, or about 398 mg to about 402 mg.

In some embodiments, provided methods comprise administering to a patient in need thereof Compound 1 in an amount that is equivalent to about 3 mg/kg, about 5 mg/kg, about 10 mg/kg, about 15 mg/kg, or about 30 mg/kg in a mouse. In some embodiments, provided methods comprise administering to a patient in need thereof Compound 1 in an amount that is equivalent to about 100 mg/kg in a mouse. In some such embodiments, the amount of Compound 1 is about 15 mg, about 25 mg, about 50 mg, about 75 mg, about 150 mg, or about 500 mg.

In some embodiments, provided methods comprise administering to a patient in need thereof Compound 1 in an amount that is equivalent to about 5 mg/kg, about 20 mg/kg, about 30 mg/kg, or about 100 mg/kg in a rat. In some such embodiments, the amount of Compound 1 is about 50 mg, about 200 mg, about 300 mg, or about 1000 mg.

In some embodiments, provided methods comprise administering to a patient in need thereof Compound 1 in an amount that is equivalent to about 5 mg/kg, about 50 mg/kg, about 150 mg/kg or about 375 mg/kg in a monkey. In some such embodiments, the amount of Compound 1 is about 100 mg, about 1000 mg, about 3000 mg, or about 7500 mg.

In some embodiments, the present invention provides a use of Compound 1 in the manufacture of a medicament for treating a MK2-mediated disease or disorder. In some embodiments, the present invention provides a use of Compound 1 for treating a MK2-mediated disease or disorder. In some such embodiments, the MK2-mediated disease or disorder us selected from ankylosing spondylitis, rheumatoid arthritis, psoriatic arthritis and psoriasis.

In some embodiments, Compound 1, or a pharmaceutically acceptable composition thereof, is administered once daily (“QD”). In some embodiments, Compound 1, or a pharmaceutically acceptable composition thereof, is administered twice daily (“BID”). In some embodiments, Compound 1, or a pharmaceutically acceptable composition thereof, is administered three times a day (“TID”). In some embodiments, Compound 1, or a pharmaceutically acceptable composition thereof, is administered four times a day (“QID”).

In some embodiments, Compound 1, or a pharmaceutically acceptable composition thereof, is administered once weekly (“QW”).

In some embodiments, provided methods comprise administering Compound 1, or a pharmaceutically acceptable composition thereof, once daily for a period of 1, 2, 3, 4, 5, 6, 7, 8, 9, 10, 11, 12, 13, 14, 15, 16, 17, 18, 19, 20, 21, 22, 23, 24, 25, 26, 27 or 28 days. In some embodiments, a pharmaceutically acceptable composition comprising Compound 1, or a pharmaceutically acceptable composition thereof, is administered once daily for 28 consecutive days (“a 28-day cycle”). In some embodiments, a pharmaceutically acceptable composition comprising Compound 1, or a pharmaceutically acceptable composition thereof, is administered once daily for at least one 28-day cycle. In some embodiments, a pharmaceutically acceptable composition comprising Compound 1, or a pharmaceutically acceptable composition thereof, is administered once daily for at least two, at least three, at least four, at least five or at least six 28-day cycles. In some embodiments, a pharmaceutically acceptable composition comprising Compound 1, or a pharmaceutically acceptable composition thereof, is administered once daily for at least seven, at least eight, at least nine, at least ten, at least eleven or at least twelve 28-day cycles. In some embodiments, a pharmaceutically acceptable composition comprising Compound 1, or a pharmaceutically acceptable composition thereof, is administered once daily for at least thirteen, at least fourteen, at least fifteen, at least sixteen, at least seventeen, at least eighteen, at least nineteen or at least twenty 28-day cycles. In some embodiments, a pharmaceutically acceptable composition comprising Compound 1, or a pharmaceutically acceptable composition thereof, is administered to a patient for the duration of the patient's life.

In some embodiments, two adjacent 28-day cycles may be separated by a rest period. Such a rest period may be one, two, three, four, five, six, seven or more days during which time the patient is not administered Compound 1, or a pharmaceutically acceptable composition thereof. In a preferred embodiment, two adjacent 28-day cycles are continuous.

In some embodiments, provided methods comprise administering to a patient in need thereof Compound 1, or a pharmaceutically acceptable composition thereof, wherein the patient has failed at least one prior therapy.

In some embodiments, Compound 1 is administered according to a regimen that minimizes exposure to ultraviolet light. In some such embodiments, Compound 1 is administered according to a regimen that minimizes exposure to sunlight. In some embodiments, Compound 1 is administered according to a regimen that minimizes exposure to long wave ultraviolet A (UVA) radiation and/or short wave ultraviolet B radiation (UVB). In some embodiments, Compound 1 is administered in the evening.

### Formulations of Compound 1

In some embodiments, the present invention provides a method of treating, stabilizing or lessening the severity or progression of one or more diseases or disorders associated with MK2, wherein the method comprises administering to a patient in need thereof a pharmaceutically acceptable composition comprising Compound 1. In some embodiments, the pharmaceutically acceptable composition is in an oral dosage form. In some such embodiments, the pharmaceutically acceptable composition is in the form of a capsule.

In some embodiments, provided pharmaceutically acceptable compositions comprise Compound 1 and one or more pharmaceutically acceptable excipients, such as, for example, binders, diluents, disintegrants, wetting agents, lubricants and adsorbents.

Pharmaceutical compositions for use in the present invention may comprise one or more binders. Binders are used in the formulation of solid oral dosage forms to hold the active pharmaceutical ingredient and inactive ingredients together in a cohesive mix. In some embodiments, pharmaceutical compositions of the present invention comprise about 5% to about 50% (w/w) of one or more binders and/or diluents. In some embodiments, pharmaceutical compositions of the present invention comprise about 20% (w/w) of one or more binders and/or diluents. Suitable binders and/or diluents (also referred to as “fillers”) are known in the art. Representative binders and/or diluents include, but are not limited to, starches such as celluloses (low molecular weight HPC (hydroxypropyl cellulose), microcrystalline cellulose (e.g., Avicel), low molecular weight HPMC (hydroxypropyl methylcellulose), low molecular weight carboxymethyl cellulose, ethylcellulose), sugars such as lactose (i.e. lactose monohydrate), sucrose, dextrose, fructose, maltose, glucose, and polyols such as sorbitol, mannitol, lactitol, malitol and xylitol, or a combination thereof. In some embodiments, a provided composition comprises a binder of microcrystalline cellulose and/or lactose monohydrate.

Pharmaceutical compositions for use in the present invention may further comprise one or more disintegrants. Suitable disintegrants are known in the art and include, but are not limited to, agar, calcium carbonate, sodium carbonate, sodium bicarbonate, cross-linked sodium carboxymethyl cellulose (croscarmellose sodium), sodium carboxymethyl starch (sodium starch glycolate), microcrystalline cellulose, or a combination thereof. In some embodiments, provided formulations comprise from about 1%, to about 25% disintegrant, based upon total weight of the formulation.

Wetting agents, also referred to as bioavailability enhancers, are well known in the art and typically facilitate drug release and absorption by enhancing the solubility of poorly-soluble drugs. Representative wetting agents include, but are not limited to, poloxamers, polyoxyethylene ethers, polyoxyethylene fatty acid esters, polyethylene glycol fatty acid esters, polyoxyethylene hydrogenated castor oil, polyoxyethylene alkyl ether, polysorbates, and combinations thereof. In certain embodiments, the wetting agent is a poloxamer. In some such embodiments, the poloxamer is poloxamer 407. In some embodiments, compositions for use in the present invention comprise from about 1% to about 30% by weight of wetting agent, based upon total weight of the blended powder.

Pharmaceutical compositions of the present invention may further comprise one or more lubricants. Lubricants are agents added in small quantities to formulations to improve certain processing characteristics. Lubricants prevent the formulation mixture from sticking to the compression machinery and enhance product flow by reducing interparticulate friction. Representative lubricants include, but are not limited to, magnesium stearate, glyceryl behenate, sodium stearyl fumarate and fatty acids (i.e. palmitic and stearic acids). In certain embodiments, a lubricant is magnesium stearate. In some embodiments, provided formulations comprise from about 0.2% to about 3% lubricant, based upon total weight of given formulation.

Pharmaceutical compositions of the present invention may further comprise one or more adsorbents. Representative adsorbents include, but are not limited to, silicas (i.e. fumed silica), microcrystalline celluloses, starches (i.e. corn starch) and carbonates (i.e. calcium carbonate and magnesium carbonate). In some embodiments, provided formulations comprise from about 0.2% to about 3% adsorbent, based upon total weight of given formulation.

In some embodiments, the present invention provides a pharmaceutical composition comprising Compound 1, methyl cellulose and Tween 80. In some such embodiments, the pharmaceutical composition is a spray-dried dispersion (SDD).

In some embodiments, provided pharmaceutical compositions comprise Compound 1, HPMCAS, microcrystalline cellulose, croscarmellose sodium, silicon dioxide, and magnesium stearate.

In some embodiments, provided pharmaceutical compositions comprise a unit dose of Compound 1. A person of ordinary skill will appreciate that the unit dosage forms described herein refer to an amount of Compound 1 as a free base. A person skilled in the art will further appreciate that, when a pharmaceutical composition comprises a salt form of Compound 1, the amount of the salt form present in the composition is an amount that is equivalent to a unit dose of the free base of Compound 1. In some embodiments, a unit dose comprises about 3 mg, about 10 mg, about 15 mg, about 25 mg, about 50 mg, about 75 mg, about 100 mg, about 125 mg, about 150 mg, about 175 mg, or about 200 mg of Compound 1. In some embodiments, a unit dose comprises about 3 mg, about 5 mg, about 10 mg, about 15 mg, about 25 mg, about 50 mg, about 75 mg, or about 100 mg of Compound 1. In some embodiments, a unit dose of Compound 1 comprises about 30 mg. In some embodiments, a unit dose of Compound 1 comprises about 60 mg. In some embodiments, a unit dose of Compound 1 comprises about 150 mg.

In some embodiments, the present invention provides a method of treating a disease or disorder associated with MK2, wherein the method comprises administering to a patient in need thereof a unit dose of Compound 1, wherein the unit dose of Compound 1 is about 3 mg, about 5 mg, about 10 mg, about 15 mg, about 25 mg, about 50 mg, about 75 mg, or about 100 mg. In some embodiments, the present invention provides a method of treating a disease or disorder associated with MK2, wherein the method comprises administering to a patient in need thereof a unit dose of Compound 1, wherein the unit dose of Compound 1 is about 30 mg, about 60 mg, or about 150 mg.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1

### Irreversible Covalent Bonding of Compound 1 to MK2

The formation of an irreversible covalent bond to human recombinant MK2 by Compound 1 to cysteine 140 (Cys140), the targeted amino acid residue in the adenosine triphosphate binding site, was investigated.

Full-length histidine-tagged human recombinant MK2 was incubated with a 10-fold molar excess of Compound 1 relative to the kinase for 1 hour at room temperature and subsequently analyzed by matrix-assisted laser desorption/ionization (MALDI) mass spectrometry to assess covalent modification. The observed centroid mass shift in the presence of Compound 1 was close to the molecular weight of Compound 1, thus confirming modification of MK2 (Table 1). Therefore, Compound 1 formed a covalent bond with human recombinant MK2.

TABLE 1
Summary of Mass Spectrometric Analysis of MK2
Protein Digest After Incubation with Compound 1

| | | | Theoretical | Observed |
|---|---|---|---|---|
| | Target | Compound | Mass of | Mass of |
| | Peptide | Exact Mass | Modified | Modified |
| Target Peptide^{a} | Mass (Da) | (Da) | Peptide (Da) | Peptide (Da) |
| ¹³³CLLIVMECLDGGELFSR¹⁴⁹ | 1897.92 | 469.09 | 2388.78 | 2388.43 |

C = cysteine; Cys140 = underlined; D = aspartic acid; Da = daltons; E = glutamic acid; F = phenylalanine; G = glycine; I = isoleucine; L = leucine; M = methionine; R = arginine; S = serine; V = valine.
^{a}Numbering corresponds to the position of amino acid residue in the full-length protein.

### Example 2

### Biochemical Potency of Compound 1 Against Recombinant MK2

Measurement of biochemical potency parameters (apparent IC₅₀ and apparent k_{inact}/K_{I}) of the inhibitory activity of Compound 1 against MK2 was performed using a continuous-read Omnia® kinase assay, which monitors kinase reaction activity and inhibition in real time. Compound 1 was tested in triplicate to determine the IC₅₀ and the overall potency (k_{inact}/K_{I}) values for its inhibition of MK2.

Results demonstrated that Compound 1 is a potent inhibitor of MK2 with an IC₅₀ value of 156.3±5.5 nM (Table 2). The inhibition is time-dependent, with an overall potency described by the kinetic parameter k_{inact}/K_{I} of (4.94±0.63)×103 M⁻¹s⁻¹.

TABLE 2
Biochemical Inhibitory Potency of Compound 1 Against MK2
Potency Against MK2: Mean ± SD

| IC₅₀ (nM) | k_{inact}/K_{I} (M⁻¹s⁻¹) |
|---|---|
| 156.3 ± 5.5 | (4.94 ± 0.63) × 103 |

IC₅₀ = concentration of inhibitor that inhibits enzyme activity 50%; K_{I} = apparent reversible dissociation constant; k_{inact} = apparent inactivation constant; k_{inact}/K_{I} = overall potency of an irreversible inhibitor; MK2 = mitogen activated protein kinase-activated protein kinase 2; SD = standard deviation.

### Example 3

### Potency of Compound 1—Inhibition of Cellular MK2

The in vitro cellular activity of Compound 1 on MK2 modulation was explored using 2 different approaches in the human THP-1 monocytic cell line: 1) measurement of inhibition of phosphorylation (Serine 78) of Hsp27, an immediate downstream target of MK2 phosphorylation and 2) determination of “free” or unbound MK2 after compound exposure. The percentage of MK2 protein not bound by Compound 1 is referred to as % free MK2.

Compound 1 demonstrated potent inhibition of cellular MK2 as measured by reduced phosphorylation of Hsp27 and by a reduction in the amount of free MK2 in human THP-1 monocytic cells with EC₅₀ values of 89±2.6 nM and of 164±18 nM, respectively.

### Example 4

### In Vivo Pharmacodynamics

In vivo, Compound 1 showed pharmacologic activity in mouse models of collagen antibody-induced arthritis (CAIA), imiquimod-induced ear swelling, and mannan-induced psoriatic arthritis disease and in an HLA-B27 transgenic model of AS in rats (Table 3). In each disease model, steady-state pharmacokinetics were characterized. Occupancy of MK2 was also quantified in spleen homogenates and in peripheral blood mononuclear cells (PBMC), and contributed towards early PK/occupancy model development.

TABLE 3
Summary of Rodent Pharmacology Studies Evaluating Compound 1

| | | Minimally | | | | |
|---|---|---|---|---|---|---|
| Disease | | Efficacious | | | AUC | % MK2 |
| Model | Endpoint | Dose | | % Inhibition | (hr · ng/mL) | Occupied |
| CAIA in | Paw swelling | 3 | mg/kg | 89.7% paw | 626 | Spleen: 28 ± 16^{c} |
| BALB/c mice | | | | swelling | | PBMC: 51 ± 16^{c} |
| Imiquimod | Ear thickness | 3 | mg/kg | 57% by ear | 917 | PBMC: 38 ± 3^{d} |
| ear swelling | | | | thickness | | |
| in BALB/c | | | | clinical score^{b} | | |
| mice | | | | | | |
| Mannan PsA | PsASI score | 3 | mg/kg | 36% by | 467 | PBMC: 47 ± 12^{d} |
| in C57B1/6 | (ear thickness, | | | PsASI^{b} | | |
| mice | skin flakiness, | | | | | |
| | and paw score | | | | | |
| | combined) | | | | | |
| Ankylosing | Paw score | 20 | mg/kg | 41.3% by paw | 2820 | Spleen: 23 ± 1.6^{c} |
| spondylitis in | | | | swelling | | PBMC: 53 ± 8.8^{c} |
| HLA B27 Tg | | | | clinical score^{b} | | |
| rat | | | | | | |

AUC = area under the plasma concentration-time curve; CAIA = collagen antibody-induced arthritis; HLA = human leukocyte antigen; PBMC = peripheral blood mononuclear cells; PsA = psoriatic arthritis; PsASI = psoriatic arthritis score index; Tg = transgenic.
^{a}AUC was determined by measurement of Compound 1.
^{b}Ear thickness, PsASI, and HLA B27 Tg paw scores were calculated from the respective area under the ear thickness-time curve, PsASI-score time curve, and paw score over time curve for the rat ankylosing spondylitis study, respectively.
^{c}Occupancy was determined in samples collected 24 hours following the last dose of Compound 1. Occupancy was also determined at 4 and 8 hours in spleen only.
^{d}Occupancy was determined in samples collected 8 hours following the last dose of Compound 1. For each study, inhibition was determined on the last day of dosing.

### Example 5

### Animal Model of Ankylosing Spondylitis

Compound 1 was evaluated in an HLA-B27 transgenic model of AS in rats to support its evaluation in this intended patient population. This animal model of AS has been described in the literature to express multiple copies of the MEW I allele, HLA B27, an allele that is associated with AS in humans (O'Neill. HLA-B27 transgenic rats: animal model of human HLA-B27-associated disorders. Toxicol Pathol. 1997; 25(4):407-8; Turner et al. HLA-B27 misfolding in transgenic rats is associated with activation of the unfolded protein response. J Immunol. 2005 Aug. 15; 175(4):2438-48; van Tok et al. Anti-IL-17A treatment blocks inflammation, destruction and new bone formation in experimental spondyloarthritis in HLA-B27 transgenic rats [abstract]. Presented at: The 2015 ACR/ARHP Annual Meeting. 2015 Nov. 6-11; San Francisco, Calif.; USA: Abstract 981. Available from: http://acrabstracts.org/abstract/anti-i1-17a-treatment-blocks-inflammation-destruction-and-newbone-formation-in-experimental-spondyloarthritis-in-hla-b 27-transgenic-rats. [Accessed 15 Dec. 2017]; van Tok et al. Innate immune activation can trigger experimental spondyloarthritis in HLA-B27/Huβ2m transgenic rats. Front Immunol. 2017 Aug. 7; 8:920). Disease-modifying activity observed in human clinical trials with anti-TNF (Liu et al. Efficacy and safety of TNF-α inhibitors for active ankylosing spondylitis patients: multiple treatment comparisons in a network meta-analysis. Sci Rep. 2016; 6:32768) and anti-IL17 (Baeten et al. Secukinumab, an interleukin-17A inhibitor, in ankylosing spondylitis. N Engl Med. 2015; 373(26):2534-48) monoclonal antibodies (mAb) have been described in this rat model of spondyloarthropathy (van Tok et al. Anti-IL-17A treatment blocks inflammation, destruction and new bone formation in experimental spondyloarthritis in HLA-B27 transgenic rats [abstract]. Presented at: The 2015 ACR/ARHP Annual Meeting. 2015 Nov. 6-11; San Francisco, Calif.; USA: Abstract 981. Available from: http://acrabstracts.org/abstract/anti-i1-17a-treatment-blocks-inflammation-destruction-and-newbone-formation-in-experimental-spondyloarthritis-in-hla-b27-transgenic-rats. [Accessed 15 Dec. 2017]; van Tok et al. Innate immune activation can trigger experimental spondyloarthritis in HLA-B27/Huβ2m transgenic rats. Front Immunol. 2017 Aug. 7; 8:920). The Janus kinase (JAK) inhibitor, tofacitinib, has also been described to have clinical activity in AS (van der Heijde et al. Tofacitinib in patients with ankylosing spondylitis: a phase II, 16-week, randomised, placebo-controlled, dose-ranging study. Ann Rheum Dis. 2017 August; 76(8):1340-7).

Etanercept and tofactinib (Balagué et al. Profiling of dihydroorotate dehydrogenase, p38 and JAK inhibitors in the rat adjuvant-induced arthritis model: a translational study. Br J Pharmacol. 2012 June; 166(4):1320-32) were included in this rat study as comparator molecules. Compound 1, tofacitinib, and vehicle were administered daily by oral gavage; etanercept was administered twice weekly as a subcutaneous injection.

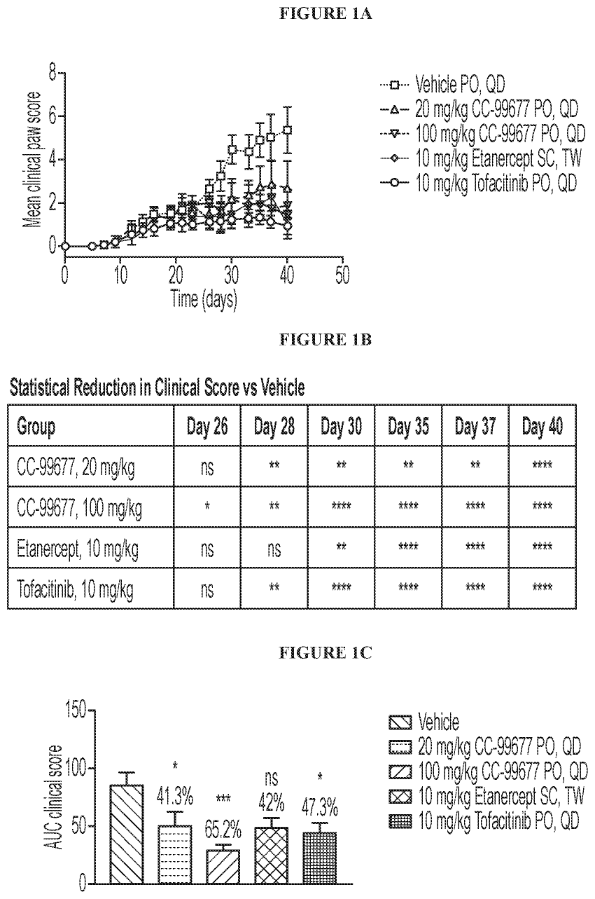

Compound 1 elicited dose-dependent inhibition of paw swelling in the HLA-B27/Huβ2m rat model of AS (Table 4; FIG. 1). The lowest tested dose determined to be effective was 20 mg/kg, which corresponded to an AUC_{LST} of 2820 ng·hr/mL and 23% and 53% target occupancy in spleen and PBMC, respectively, at 24 hours. A trend towards decreases in the cytokines IL-7 and IL-1β; the chemokines MCP-1, macrophage inflammatory protein-1 alpha (MIP-1α), keratinocyte chemoattractant (KC); and granulocyte macrophage colony-stimulating factor (GM-CSF) were observed.

TABLE 4
Disease Inhibition, MK2 Occupancy, and Compound
1 Plasma Exposure in Male HLA-B27/Huβ2m Rats

| | | | Mean PBMC | Mean Spleen | |
|---|---|---|---|---|---|
| | Dose | Paw Swelling | Occupancy (%) | Occupancy (%) | AUC_{LST} |
| Treatment | (mg/kg) | Inhibition (%) | (mean ± SD) | (mean ± SD) | (ng · hr/mL) |
| Naive | N/A | N/A | N/A | N/A | N/A |
| Vehicle | N/A | N/A | N/A | N/A | N/A |
| Compound 1 | 20 | 41 | 53 ± 8.8 | 23 ± 1.6 | 2820 (160)^{a} |
| Compound 1 | 100 | 65 | 72 ± 3.7 | 30 ± 5.6 | 7190 (468)^{a} |
| Tofacitinib | 10 | 47 | N/A | N/A | 6540 |
| Etanercept | 10 | 42 | N/A | N/A | ND |

AUC_{LST} = area under the concentration-time curve from time zero to the last quantified timepoint following dosing; N/A = not applicable; ND = not determined; PBMC = peripheral blood mononuclear cells; SD = standard deviation.
N = 3 for PBMC and spleen occupancy.

### Example 6

### Nonclinical Pharmacokinetics and Metabolism

In vitro and in vivo studies were conducted to characterize the absorption, PK, distribution, and metabolism of Compound 1. Robust and reproducible bioanalytical methods for the determination of plasma concentrations of Compound 1 were developed and used in PK and GLP-compliant toxicokinetic (TK) studies. Pharmacokinetics and oral bioavailability of Compound 1 were evaluated in mice and monkeys, the rodent and non-rodent species used for nonclinical toxicity assessment, as well as in rats. In vitro studies were conducted to assess Compound 1 metabolism, plasma protein binding, blood to plasma partitioning, the human enzymes responsible for metabolism, and the inhibition and induction potential of CYP enzymes. Additionally, plasma samples from single dose PK studies in mice and monkeys were evaluated for circulating metabolites.

The pharmacokinetics of Compound 1 were evaluated in male and female CD-1 mice, Sprague Dawley rats, and cynomolgus monkeys following intravenous (IV) and oral administration. The mean PK parameters are summarized in Table 5 and Table 6.

The systemic clearance of Compound 1 was low (approximately 25% of liver blood flow) in mice, moderate in rats (approximately 29% to 56% of liver blood flow), and high (similar to liver blood flow) in monkeys. The volume of distribution was high in mice and monkeys (>2- to 3-fold total body water volume) and moderate in rats (similar to body-water volume). The terminal half-life of Compound 1 was long in mice (approximately 7 hours) and short in rats and monkeys (≤1 hour). Following oral dosing, Compound 1 was absorbed with a time to maximum plasma concentration (tmax) of 0.25 to 1 hour in mice and rats and 1 to 6 hours in monkeys. The oral bioavailability of Compound 1 was low in rats and monkeys (8% to 28%) and low to moderate in mice (22% to 51%) at doses in the range 15 to 375 mg/kg.

Several single-dose PK and TK studies were conducted in cynomolgus monkeys using various excipients and formulation approaches to identify the optimal formulation for toxicology studies. An SDD formulation containing 35% Compound 1/65% HPMCAS was chosen for further development of Compound 1. This formulation could support the high dose concentrations required for toxicology studies, as well as providing high plasma exposures. The maximum feasible dose concentration of Compound 1 SDD in water was 75 mg/mL, which supported a maximum feasible dose of 750 mg/kg and 375 mg/kg in mouse and monkey 28-day GLP-compliant toxicology studies, respectively.

In the 28-day GLP-compliant toxicology study in mice with Compound 1, systemic exposure (AUC_{LST}) increased in an approximately dose-proportional manner in males and females for Compound 1 on Day 1 and in a less than dose-proportional manner on Day 21 in the Compound 1 dose range of 15 to 750 mg/kg/day. Upon repeated Compound 1 dosing, the Day 21 to Day 1 AUC_{LST} ratios for Compound 1 suggested modest time-dependent decreases in systemic exposure to Compound 1 at 750 mg/kg, but no time-dependent changes at the lower doses tested. There were no notable sex-related differences in Compound 1 exposure.

In the 28-day GLP-compliant toxicology study in monkeys with Compound 1, systemic exposure (AUC_{LST}) for Compound 1 increased in an approximately dose-proportional manner on Day 1 and in a less than dose-proportional manner on Day 20 in the Compound 1 dose range of 50 to 375 mg/kg/day. There was a reduction in Compound 1 exposure at steady-state (AUC_{LST} ratios of 0.225 to 1.25), which was higher in the highest dose group (375 mg/kg) compared to the other dose groups.

TABLE 5
Mean Plasma Pharmacokinetic Parameters Following a Single Intravenous
Dose of Compound 1 to Animals

| | | Dose^{a} | AUC_{LST} | AUC_{∞} | CL | Vdₛₛ | t_{1/2,z} |
|---|---|---|---|---|---|---|---|
| Species | Sex | (mg/kg) | (ng · h/mL) | (ng · h/mL) | (mL/h/kg) | (mL/kg) | (h) |
| Mouse | Male | 5 | 4160 | 4350 | 1150 | 5080 | 7.3 |
| | Female | 5 | 3400 | 3430 | 1460 | 2490 | 6.6 |
| Rat | Male | 5 | 2020 | 2020 | 2530 | 648 | 0.23 |
| | | | (375) | (381) | (430) | (25.0) | (0.0058) |
| | Female | 5 | 3950 | 3960 | 1290 | 561 | 1.1 |
| | | | (663) | (663) | (234) | (31.2) | (0.73) |
| Monkey | Male | 5 | 1920 | 1920 | 2610 | 1810 | 0.95 |
| | | | (114) | (114) | (157) | (277) | (0.57) |
| | Female | 5 | 2200 | 2210 | 2300 | 1930 | 0.98 |
| | | | (352) | (349) | (332) | (278) | (0.37) |

AUC_{LST} = area under the concentration-time curve from time zero to the last quantifiable timepoint following dosing;
AUC_{∞} = area under the concentration-time curve extrapolated from time 0 to infinity;
CL = clearance;
SD = standard deviation;
t_{1/2,z} = apparent half-life of the terminal phase of the concentration-time curve;
Vdₛₛ = volume of distribution at steady state.
^{a}Doses shown are free base equivalents.
Pharmacokinetic parameters from rats and monkeys are from n = 3 animals/sex/group and values shown are mean and SD (in parentheses).
For mice, composite PK parameters were determined using average plasma concentrations from 3 animals/sex/group/time point.

TABLE 6
Mean Plasma Pharmacokinetic Parameters Following a Single Oral Dose of Compound 1 to Animals

| | | Dose^{a} | tₘₐₓ | Cₘₐₓ | AUC_{LST} | AUC_{∞} | F |
|---|---|---|---|---|---|---|---|
| Species | Sex | (mg/kg) | (h) | (ng/mL) | (ng · h/mL) | (ng · h/mL) | (%) |
| Mouse | Male | 15 | 0.25 | 1270 | 2870 | 3040 | 23 |
| | Female | 15 | 0.50 | 1980 | 5170 | 5250 | 51 |
| | Male | 100 | 0.50 | 6600 | 19200 | 19200 | 22 |
| | Female | 100 | 0.25 | 8080 | 28200 | 28400 | 41 |
| Rat | Male | 30 | 0.25 | 1180 | 1190 | 1290 | 11 |
| | | | (0.25-0.50) | (511) | (434) | (395) | (3) |
| | Female | 30 | 0.50 | 4960 | 5940 | 6610 | 28 |
| | | | (0.25-1.0) | (1270) | (3530) | (3510) | (15) |
| Monkey | Male | 50 | 2.0 | 865 | 3680 | 3710 | 18 |
| | | | (1.0-2.0) | (179) | (820) | (849) | (4) |
| | Female | 50 | 2.0 | 1040 | 3810 | 3840 | 19 |
| | | | (2.0-2.0) | (634) | (1770) | (1780) | (9) |
| | Male | 150 | 1.0 | 1990 | 8480 | 8590 | 14 |
| | | | (1.0-1.0) | (604) | (1750) | (1830) | (3) |
| | Female | 150 | 2.0 | 1200 | 4750 | 4820 | 8 |
| | | | (1.0-4.0) | (728) | (2160) | (2190) | (4) |
| | Male | 375 | 2.0 | 1890 | 13900 | 14000 | 9 |
| | | | (1.0-6.0) | (712) | (3590) | (3590) | (2) |
| | Female | 375 | 6.0 | 2530 | 22600 | 22700 | 15 |
| | | | (1.0-6.0) | (750) | (7780) | (7810) | (5) |

AUC_{LST} = area under the concentration-time curve from time zero to the last quantified timepoint following dosing;
AUC_{∞} = area under the plasma concentration-time curve extrapolated from time 0 to infinity;
Cₘₐₓ = maximum plasma concentration;
F = oral bioavailability;
SD = standard deviation;
tₘₐₓ = time of Cₘₐₓ.
^{a}Doses shown are free base equivalents.
Pharmacokinetic parameters are from n = 3 animals/sex and values shown are mean and SD (in parentheses), except for tₘₐₓ where values shown are median and range (in parentheses).

### Example 7

### Toxicity Studies

Initial exploratory non-pivotal toxicity studies were conducted with Compound 1 administered as a SDD in 0.5% methyl cellulose (400 cps) and 0.1% Tween 80 (Table 7).

A single oral dose study with a 14-day repeat-dose phase was conducted in male and female mice. 250 mg/kg was selected as the highest dose for the repeat dose portion of the study. After 14 days of dosing in mice at up to 250 mg/kg/day, no compound related findings were identified.

In cynomolgus monkeys, a 7-day repeat dose toxicity study was conducted with doses up to 250 mg/kg/day. There were no compound-related findings in any endpoints. To aid in dose selection for a subsequent pivotal study, an additional study was conducted to determine the maximum feasible dose under optimal feeding conditions in cynomolgus monkeys. Compound 1 SDD was administered as single doses of 375 mg/kg in 0.5% methyl cellulose (400 cps) and 0.1% Tween 80 (dose formulation concentration of 75 mg/mL), or 500 mg/kg in distilled water (dose formulation concentration of 100 mg/mL) to fasted or non-fasted cynomolgus monkeys. Exposure was similar at both the 375 mg/kg and 500 mg/kg dose and, in addition, it was noted that the 100 mg/mL formulation concentration required to administer the higher dose of 500 mg/kg was too viscous to allow for consistent dose administration in a repeat-dose study. Dosing monkeys in the fasted state prior to dosing yielded more consistently higher exposures. Therefore, a dose of 375 mg/kg in 0.5% methyl cellulose (400 cps) and 0.1% Tween 80 administered to fasted monkeys was selected for the 14-day repeat-dose phase of the study, and was considered the maximum feasible dose.

The results of these studies were used to select doses for the GLP-compliant repeat-dose toxicity studies. None of the exploratory repeat-dose toxicity studies identified any compound-related findings. Therefore, the maximum feasible dose was selected as the high dose for the pivotal repeat-dose toxicity studies. Due to formulation limitations, the maximum feasible dose was determined to be 750 mg/kg for mice and 375 mg/kg for monkeys.

Additionally, the vehicle used in the pivotal studies was changed from 0.5% methyl cellulose (400 cps) and 0.1% Tween 80 to distilled water. When Tween 80 was present in the vehicle, Compound 1 changed from an amorphous to a crystalline form, beginning within the first hour after dose formulation preparation. Conversion to the crystalline form did not occur when Compound 1 was prepared in distilled water and all Compound 1 remained in the amorphous form for at least 24 hours. The amorphous form of Compound 1 yields higher bioavailability of the molecule, and therefore, a water vehicle allowed for maximum Compound 1 exposure in the test species.

TABLE 7
Toxicity Studies

| | Dosing | Doses | Sex/No. per | |
|---|---|---|---|---|
| Species/Strain | Duration | (mg/kg/day) | Group | Findings |
| Mouse/CD-1 | Single dose | Single dose: | Single dose: | Single dose phase: |
| | and 14-day | 50, 250, 750 | 9M/9F | Piloerection at 50 and |
| | repeat dose | 14-day repeat: | 14-day repeat: | 750 mg/kg |
| | phases | 0, 15, 75, 250 | 10M/10F | Cold to touch and shaking |
| | | | | at 750 mg/kg |
| | | | | 14-day phase: |
| | | | | No compound-related findings |
| | | | | Mean Day 14 AUC₀₋ₗₐₛₜ TK |
| | | | | parameters: |
| | | | | 15 mg/kg/day: |
| | | | | 4820 hr · ng/mL |
| | | | | 75 mg/kg/day: |
| | | | | 16,600 hr · ng/mL |
| | | | | 250 mg/kg/day: |
| | | | | 35,100 hr · ng/mL |
| Monkey/Cynomolgus | 7 days | 0, 25, 75, 250 | 2M/2F | No compound-related findings |
| | | | | Mean Day 7 AUC₀₋ₗₐₛₜ TK |
| | | | | parameters: |
| | | | | 25 mg/kg/day: |
| | | | | 1090 hr · ng/mL |
| | | | | 75 mg/kg/day: |
| | | | | 1169 hr · ng/mL |
| | | | | 250 mg/kg/day: |
| | | | | 9435 hr · ng/mL |
| Monkey/Cynomolgus | Single dose | Single dose | Single dose: | Single dose phase: |
| | and 14-day | fasted and | 3M | No compound-related findings |
| | repeat dose | non-fasted: | 14-day repeat: | Mean Day 1 AUC₀₋ₜ TK |
| | phases | 375 (0.5% | 3M | parameters: |
| | | methylcellulose/ | | 375 mg/kg, non-fasted: |
| | | 0.1% Tween 80 | | 4060 hr · ng/mL |
| | | vehicle) and | | 375 mg/kg, fasted: |
| | | 500 (distilled | | 11,500 hr · ng/mL |
| | | water vehicle) | | 500 mg/kg, non-fasted: |
| | | 14-day repeat | | 11,400 hr · ng/mL |
| | | fasted: 0, 375 | | 500 mg/kg, fasted: |
| | | | | 14300 hr · ng/mL |
| | | | | No compound-related findings |
| | | | | 14-day phase: |
| | | | | Mean Day 14 AUC₀₋ₜ TK |
| | | | | parameters: |
| | | | | 375 mg/kg/day: 5510 hr · ng/mL |

AUC₀₋ₗₐₛₜ = area under the plasma concentration time curve from time 0 to the last time point with quantifiable concentration; AUC₀₋ₜ = area under the plasma concentration time curve from time 0 to the last time point with quantifiable concentration; F = females; M = males; TK = toxicokinetic.

### Example 8

### 28-Day Repeat-Dose Oral Toxicity Study in Mice

Compound 1, supplied as an SDD containing 35% w/w Compound 1 in HPMCAS-M was administered via oral gavage to male and female Crl:CD1® (ICR) mice at dosage levels of 0 (deionized water), 15, 100, or 750 mg/kg/day for a period of 28 consecutive days. Additionally, toxicokinetic animals received the vehicle or compound in the same manner and dose levels as the main study groups.

All animals survived to the scheduled necropsies.

There were no compound-related effects on clinical observations, food consumption, body weight, ophthalmic examination findings, serum chemistry and urinalysis parameters, or macroscopic observations.

There were no compound-related findings at 15 mg/kg/day.

The following changes were considered the result of Compound 1 administration:

Adverse compound-related effects:
Minimal to mild myofiber degeneration in the heart at 750 mg/kg/day

Non-adverse compound-related effects:
Minimal to mild decreases in lymphocyte counts and inconsistent decreases in most other leukocyte subtypes, including neutrophil (females only), monocyte (females only), eosinophil (males only) and basophil counts, relative to controls at 750 mg/kg/day, with decreases in lymphocyte count correlating with lymphoid depletion in the thymus
Mild increase in mean platelet count (+96%), and a mild decrease in mean absolute reticulocyte count (−29%), relative to controls in males at 750 mg/kg/day
Increased liver and heart weights relative to controls at 750 mg/kg/day, with the higher liver weights of males correlating microscopically with Compound 1-related hepatocellular hypertrophy
Compound 1-related microscopic findings in the liver, skeletal muscle, duodenum, and thymus:
   Centrilobular hepatocellular hypertrophy (typically of minimal severity) at 750 mg/kg/day
   Minimal to mild myofiber degeneration in the skeletal muscle at 100 mg/kg/day, predominantly observed in the soleus and quadriceps muscles, characterized by scattered myofibers with evidence of degeneration (fragmentation and/or macrophage infiltration) and a large percentage of regenerating myofibers (basophilic cytoplasm with central or rowed nuclei).
   Minimal to mild erosion/ulceration of the proximal duodenum at 750 mg/kg/day
   Minimal to mild thymic cortical lymphoid depletion, which correlated to decreases in lymphocyte count at 750 mg/kg/day

A summary of toxicokinetic parameters are included in Table 8 for Compound 1.

TABLE 8
Summary of Mean Toxicokinetic Parameters for Compound 1 in Mice

| Dose | Male | | | Female | | |
|---|---|---|---|---|---|---|
| (mg/kg/day) | 15 | 100 | 750 | 15 | 100 | 750 |

Study Day 1

| | | | | | | |
|---|---|---|---|---|---|---|
| Cₘₐₓ | 2930 | 13900 | 29800 | 2980 | 14500 | 50300 |
| (ng/mL) | | | | | | |
| AUC_{LST} | 4410 | 29200 | 211000 | 5360 | 40100 | 198000 |
| (ng · h/mL) | | | | | | |

Study Day 21

| | | | | | | |
|---|---|---|---|---|---|---|
| Cₘₐₓ | 2420 | 11700 | 19400 | 2970 | 12300 | 23200 |
| (ng/mL) | | | | | | |
| AUC_{LST} | 4460 | 24900 | 92800 | 5980 | 32800 | 135000 |
| (ng · h/mL) | | | | | | |

AUC_{LST} = area under the plasma concentration-time curve from time zero to the time of the last quantifiable concentration;
Cₘₐₓ = maximum plasma concentration.

There were no notable sex differences in Compound 1 exposure. Average (sexes combined) exposure (AUC_{LST}) to Compound 1 increased in an approximately dose-proportional manner on Day 1 (42.4-fold increase) and in a less than dose-proportional manner on Day 21 (21.7-fold increase) in the Compound 1 dose range of 15 to 750 mg/kg/day (50-fold increase).

Based upon adverse findings in the heart at 750 mg/kg/day, the NOAEL was established at 100 mg/kg/day, corresponding to an AUC_{LST} of 28,900 and 10,300 ng·hr/mL for

Compound 1.

### Example 9

### Pivotal 28-Day Repeat-Dose Toxicity Study in Monkeys

Compound 1, supplied as an SDD that contains 35% w/w Compound 1 in HPMCAS-M, was administered via oral gavage to male and female cynomolgus monkeys (Macaca fascicularis) (3/sex/group) at dosage levels of 0 (deionized water), 50, 150, and 375 mg/kg/day for a period of 28 consecutive days.

There were no compound-related effects on mortality, body weight, ophthalmic examinations, electrocardiograms, clinical pathology parameters, macroscopic observations, or organ weights.

There were no adverse compound-related findings.

Non-adverse compound-related effects included:
Clinical observations limited to dose-dependent incidents of discolored urine at ≥150 mg/kg/day and discolored and/or watery stool at ≥50 mg/kg/day
Minimal myofiber degeneration in skeletal muscle at ≥150 mg/kg/day at a higher incidence compared to controls, characterized by rare scattered myofibers with evidence of degeneration (cytoplasmic hypereosinophilia, loss of cross striations, or fragmentation) and variable evidence of regeneration (cytoplasmic basophilia and central or rowed nuclei) and most commonly observed in the psoas muscle, and at a lower incidence, in the rectus femoris muscle.

A summary of toxicokinetic parameters are included in Table 9 for Compound 1:

TABLE 9
Summary of Mean Toxicokinetic Parameters for
Compound 1 in Monkey

| Dose | Male | | | Female | | |
|---|---|---|---|---|---|---|
| (mg/kg/day) | 15 | 150 | 375 | 15 | 150 | 375 |

Study Day 1

| | | | | | | |
|---|---|---|---|---|---|---|
| Cₘₐₓ | 623 | 1240 | 1960 | 862 | 1390 | 2980 |
| (ng/mL) | | | | | | |
| AUC_{LST} | 1750 | 5840 | 14100 | 3090 | 6330 | 16700 |
| (ng · h/mL) | | | | | | |

Study Day 21

| | | | | | | |
|---|---|---|---|---|---|---|
| Cₘₐₓ | 465 | 674 | 2340 | 818 | 1620 | 810 |
| (ng/mL) | | | | | | |
| AUC_{LST} | 1280 | 3950 | 8790 | 2460 | 7840 | 4220 |
| (ng · h/mL) | | | | | | |

AUC_{LST} = area under the plasma concentration-time curve from time zero to the time of the last quantifiable concentration;
Cₘₐₓ = maximum plasma concentration.

The male and female average systemic exposure (mean AUC_{LST}) for Compound 1 increased in an approximately dose-proportional manner on Day 1 (6.73-fold increases) and in a less than dose-proportional manner on Day 20 (4.29-fold increases) in the Compound 1 dose range of 50 to 375 mg/kg/day (7.5-fold increase).

Based upon the lack of adverse changes in any parameter evaluated, the NOAEL for Compound 1 with respect to dose was 375 mg/kg/day, the highest dose used in the study. The NOAEL for Compound 1 with respect to AUC_{LST} was 8790 ng·hr/mL (375 mg/kg/day) for males and 7840 ng·hr/mL (150 mg/kg/day) for females, the highest systemic exposures achieved at steady-state in the study.

### Example 10

A phase 1, randomized, single-center, 3-part, first in human (FIH) study was conducted to assess the safety, tolerability, PK, and pharmacodynamics (PD) of single and multiple doses of Compound 1 and to characterize the effect of food on the single dose PK of Compound 1 in healthy adult subjects.

For Parts 1 and 2 the study participants, Investigators, and any other site staff directly involved in the conduct of the trial were blinded to treatment throughout the study. Part 3 will be conducted in an open label fashion.

Part 1. Part 1 was a randomized, double-blind, placebo-controlled study to evaluate the safety, tolerability, PK, and PD of Compound 1 following administration of single oral doses in healthy adult subjects.

Part 1 consisted of escalating single doses in sequential groups. Approximately 48 subjects were enrolled into 6 planned dose level cohorts as shown in Table 10:

TABLE 10

| | Compound 1 Dose Level | |
|---|---|---|
| Cohort | (Total Daily Dose) | |
| 1A | 3 | mg |
| 1B | 10 | mg |
| 1C | 30 | mg |
| 1D | 100 | mg |
| 1E | 200 | mg |
| 1F | 400 | mg |

Each dose level cohort consisted of 8 subjects; 6 subjects received Compound 1 and 2 subjects received placebo according to the randomization schedule. For all dose levels in Part 1, the first 2 subjects (sentinel) were randomized 1:1 to placebo or Compound 1, and the remaining 6 subjects were randomized 1:5 to placebo or Compound 1, respectively. The 2 sentinel subjects (chosen according to a randomization scheme) were dosed on Day 1. After at least 24 hours, the safety profile was acceptable to the Investigator (based on, at minimum, adverse events [AEs], concomitant medications and procedures, and any other important clinical observations), and the remainder of the cohort was dosed according to the randomization schedule.

Investigational product was administered at only 1 dose level at a time. Administration at the next dose level did not begin until the safety and tolerability of the preceding dose level was evaluated and deemed acceptable by the Investigator and Sponsor's Medical Monitor, and exposure of Compound 1 remained within the prespecified limit (area under the concentration time curve from time zero to 24 hours postdose [AUC₀₋₂₄]≈7840 ng·hr/mL, which is equivalent to the steady state exposure at the no observed adverse effect level [NOAEL] in female monkeys). There was an interval of no less than 7 days between the dosing of successive cohorts.

Investigational product (either Compound 1 or placebo) was administered on Day 1, under fasted conditions, according to the randomization schedule.

Part 2. Part 2 was a randomized, double-blind, placebo-controlled study to evaluate the safety, tolerability, PK, and PD of Compound 1 following administration of multiple oral doses (14 days) in healthy adult subjects.

Part 2 did not begin until safety and PK data from at least the first 3 dose levels in Part 1 were evaluated. Only doses that were safe and tolerated in Part 1 and predicted, at steady state, not to exceed exposures which were safe and tolerated in Part 1 and Compound 1 steady state exposure at the NOAEL in female monkeys (AUC₀₋₂₄≈7840 ng·hr/mL) were administered in Part 2.

The study consisted of escalating multiple doses in sequential groups. 37 subjects were randomized and enrolled into 5 proposed dose level cohorts.

Sentinel dosing was not proposed for any dose level in Part 2 provided that emerging PK data from Part 1 indicated exposure increases in a dose proportional or less than dose proportional manner.

Investigational product was administered at only 1 dose level at a time. Administration at the next dose level did not begin until the safety and tolerability of the preceding dose level was evaluated and deemed acceptable by the Investigator and Sponsor's Medical Monitor, and exposure of Compound 1 remained within the prespecified limit (AUC₀₋₂₄≈7840 ng·hr/mL). In Part 2, there was an interval of no less than 14 days between the dosing of successive cohorts. Additionally, a minimum of 3 weeks of safety data from the preceding dose level cohort was reviewed prior to escalation to the next dose level.

The first dose of investigational product (either Compound 1 or placebo) was administered on Day 1, under fasted conditions, according to the randomization schedule. The same total daily dose was then administered for the remainder of the planned treatment schedule (Days 2 to 14, inclusive).

Part 3. Part 3 was an open label, randomized, 2 period, 2 way crossover study to characterize the effect of food on the single dose PK of Compound 1 in healthy adult subjects.

Part 3 did not begin until safety and PK data from at least the first 5 dose levels in Part 1 were evaluated. The dose administered in Part 3 was selected based on data from Part 1. Only doses that were safe and tolerated in Part 1 and predicted not to exceed a Compound 1 exposure at the NOAEL in female monkeys (AUC₀₋₂₄≈7840 ng·hr/mL) were administered. To account for the possibility that food may have an effect on Compound 1 exposure, only doses predicted not to exceed the 7840 ng·hr/mL exposure even after a potential 4 fold increase in exposure were administered (a 4 fold increase would account for an increase from the predicted human bioavailability of 25% to 100%).

Approximately 12 subjects were assigned randomly to 1 of 2 treatment sequences. Each subject will receive Treatment A and Treatment B separated by an appropriate interval, and the randomly assigned sequences will dictate the order in which each subject receives each treatment:
Treatment A: A single dose of Compound 1 administered as a formulated capsule(s) under fasted conditions
Treatment B: A single dose of Compound 1 administered as a formulated capsule(s) under fed conditions

The study consisted of 2 study periods. Subjects received their assigned treatment on Day 1 of each study period, either under fasted or fed conditions depending on the treatment sequence to which they were randomized.

Each dose was separated by an appropriate interval (i.e., washout), which will be based on data obtained in Part 1 of the study. This washout was, at minimum, equal to or greater than five times the estimated terminal elimination half-life (t1/2,z) of Compound 1.

For doses administered under fed conditions (Treatment B), subjects received a standard high fat, high calorie breakfast approximately 30 minutes prior to dosing.

Results. This study was a Phase 1, three-part study with a double-blind, placebo-controlled, single ascending dose (SAD) phase, a double-blind multiple ascending dose (MAD) phase, and an open-label cross-over food effect phase. The study enrolled a total of 97 healthy adult subjects.

Part 1 (single ascending dose, or SAD, phase) evaluated the safety, tolerability, and PK of Compound 1 following single oral doses of 3 mg to 400 mg. In Part 1, 48 subjects were randomized and enrolled into 6 cohorts. Each cohort consisted of 8 subjects: 6 subjects received Compound 1, and 2 subjects received placebo. The most common treatment-emergent adverse events (TEAEs) in this part were in the categories of musculoskeletal and connective tissue disorders, skin and subcutaneous tissue disorders, and infections and infestations. In Part 1, 5 of the 22 total TEAEs were suspected to be related to Compound 1. The most common TEAEs suspected of being related to Compound 1 were in the skin and subcutaneous tissue disorder category with 2 TEAEs in 2 subjects who had a mild, transient, pruritic, erythematous rash without other organ involvement at the 400-mg dose level. The majority of TEAEs were mild in nature. Only one TEAE was moderate in nature (gastroenteritis lasting less than 24 hours in the 30 mg cohort). There were no severe TEAEs or serious adverse events (SAEs).

Part 2 (MAD phase) evaluated the safety, tolerability, and PK of Compound 1 following multiple daily oral doses of 10, 30, 60, 120, and 150 mg. In Part 2, 37 subjects were randomized and enrolled and received 14 days of dosing. The most common TEAEs in Part 2 were categorized under nervous system disorders (headache and dizziness) and gastrointestinal disorders (nausea, constipation, diarrhea, abdominal pain, dyspepsia, and rectal bleeding). In Part 2, 9 of the 42 total TEAEs were suspected to be related to Compound 1. The most common TEAEs suspected of being related to Compound 1 in Part 2 were in the vascular disorders class with 2 TEAEs in 2 subjects (skin flushing in 1 subject and hot flush in 1 subject). In the 120-mg dose cohort there was an observation in 3 of 6 subjects on active treatment with an increase in serum transaminase levels (alanine aminotransferase [ALT] in particular) above the upper limit of the normal reference range (ULN). The ALT elevation in one of these subjects, which peaked at 2.3×ULN at Day 17, was judged by the Investigator to be a TEAE suspected to be related to Compound 1. This AE resolved without intervention when the ALT returned toward the normal reference range by day 28. The other 2 subjects had ALT elevations less than 2× above the upper limit of normal. All subjects in the 120-mg cohort with ALT values crossing above the ULN were asymptomatic, and on follow-up laboratory testing after completion of dosing, all transaminase levels began to return toward the baseline values without intervention. No other liver chemistry abnormalities were noted. In the subsequent 150-mg dose cohort, no transaminase level changes were observed in any subject during the 14-day dosing period. One subject in the 150-mg dose cohort was found to have an elevated aspartate transaminase (AST) level on outpatient follow up 14 days after the end of dosing (Day 28), which was determined to be the result of increased exercise undertaken by the normally sedentary subject after leaving the site at the end of the confinement period. There was no apparent trend towards increasing liver transaminase concentrations (ALT and AST) in any subject in that dose cohort.

Based upon review of the safety data, including physical examinations, vital sign measurements, clinical laboratory assessments, electrocardiograms (ECGs), and TEAEs, Compound 1 appears to be well tolerated when administered as a single dose up to 400 mg and when administered for 14 days up to 150 mg.

Clinical Pharmacology. The clinical pharmacology study evaluated single doses of Compound 1 ranging from 3 to 400 mg and also repeat daily doses (QD×14 days) of Compound 1 ranging from 10 mg to 150 mg.

Following single oral dosing, Compound 1 was rapidly absorbed with a median time to observed maximum plasma concentration (Tₘₐₓ) of 1 to 3 hours across the dose range of 3 mg to 400 mg. The systemic exposure (maximum plasma concentration [Cₘₐₓ] and area under the curve [AUC]) of Compound 1 increased in an approximately dose proportional manner across this range. The terminal elimination half-life (t½) ranged from (geometric mean) 1.66 to 6.95 hours.

After multiple daily doses, Compound 1 was absorbed rapidly with a Tₘₐₓ of 1 to 2.5 hours across the dose range of 10 mg to 150 mg. The systemic exposures (Cₘₐₓ and AUC) on Day 14 of Compound 1 increased in an approximately dose-proportional manner. The t½ on Day 14 was similar across studied doses, ranging from (geometric mean) 1.96 to 4.39 hours. There was limited accumulation of Compound 1 (range of accumulation ratio for AUC₀₋₄ was 1.1 to 1.2 from 10 to 150 mg, respectively) upon repeated daily doses.

Food has limited effect on the PK of the current formulation of Compound 1. Therefore, Compound 1 can be taken with or without food.

Pharmacodynamics. The pharmacodynamics (PD) of Compound 1 were assessed during Parts 1 and 2 of the study by evaluating the effect of ascending dose levels of Compound 1 on target engagement (TE) with MK2 protein and on levels of proinflammatory cytokines using a whole-blood ex vivo assay in which blood isolated from subjects treated with Compound 1 or placebo was incubated with lipopolysaccharide stimulation (LPS) for 24 hours. A dose dependent increase in TE and TNF inhibition in the ex vivo assay was observed by 4 hours with single oral doses ranging from 10 mg to 400 mg in Part 1 of the study. Daily dosing of Compound 1 in Part 2 of the study resulted in a dose-dependent increases in TE following doses between 10 mg and 120 mg and plateauing at the 120 mg and 150 mg dose levels. This was accompanied by sustained reductions in TNF-α over 14 days of dosing, at doses greater than 10 mg, with maximal inhibition at 150 mg.

### Example 11

A Phase 2, randomized, double-blind, placebo-controlled, multicenter study will be conducted to evaluate the efficacy and safety of 2 dose groups of Compound 1 in subjects with active AS. The main study population comprises AS subjects who have failed therapy with at least 2 NSAIDs; an additional substudy will recruit AS subjects who have who have failed therapy with 2 NSAIDs and who have also failed 1 biologic agent. These patient populations were chosen to represent AS patients most likely to derive clinical benefit from Compound 1. Since biologic-failure subjects are expected to be less responsive to anti-inflammatory treatments, in general, the benefit-risk profile of Compound 1 will be evaluated separately for the main study population and the substudy population.

Efficacy assessment in this study will be based on patient and physician-reported outcomes, and objective measures of inflammation and disease activity. The ASAS Response Criteria are based on a disease assessment tool that utilizes results from a subject self-administered survey. The ASAS 20 and ASAS 40 criteria have been widely validated as tools to measure symptomatic improvement in the context of AS clinical trials. The ASAS 20 is the primary endpoint in this study and has been used extensively in most Phase 2 and 3 clinical trials in AS. Several other disease activity measures will be assessed to corroborate the efficacy evaluation of Compound 1 during the study. These include ASAS 40, ASDAS-CRP, BASDAI, Bath Ankylosing Spondylitis Functional Index (BASFI) and Bath Ankylosing Spondylitis Metrology Index-Linear (BASMI-Linear). Magnetic resonance imaging of the spine and sacroiliac joints using the Spondyloarthritis Research Consortium of Canada (SPARCC) scoring system (Maksymowych, et al. Spondyloarthritis Research Consortium of Canada Magnetic Resonance Imaging Index For Assessment of Sacroiliac Joint Inflammation in Ankylosing Spondylitis. Arthrisit Rheum. 2005; 53(5):703-9; Maksymowych, et al. Low-dose Infliximab (3 mg/kg) Significantly Reduces Spinal Inflammation on Magnetic Resonance Imaging in Patients with Ankylosing Spondylitis: A Randomized Placebo-controlled Study. J Rheumatol. 2010; 37 (8); 1728-34) will be performed to assess the effect of Compound 1 on spinal and sacroiliac joint inflammation. Magnetic resonance imaging provides an objective assessment of inflammation and evidence of sacroiliitis and predicts radiographic progression of disease (reviewed in Schwartzman, et al. Is There a Role for Mill to Establish Treatment Indications and Effectively Monitor Response in Patients with Axial Spondyloarthritis? Rheum Dis Clin North Am. 2019; 45(3):341-58). In clinical trials of AS, clinical improvements are accompanied by rapid (within 12 weeks) reduction in bone marrow edema, as measured by Mill (Lambert, et al. Adalimumab significantly reduces spinal and sacroiliac joint inflammation in patients with ankylosing spondylitis a multicenter, randomized, double-blind, placebo-controlled study. Arthritis Rheum. 2007; 56(12):4005-14; Braun, et al. Effect of certolizumab pegol over 96 weeks of treatment on inflammation of the spine and sacroiliac joints, as measured by MM, and the association between clinical and Mill outcomes in patients with axial spondyloarthritis. RAID Open 2017; 3 (1):e000430; van der Heijde, et al. Tofacitinib in patients with ankylosing spondylitis: a phase II, 16-week, randomised, placebo controlled, dose-ranging study. Ann Rheum Dis. 2017; 76(8):1340-47; van der Heijde, et al. Efficacy and safety of filgotinib, a selective Janus kinase 1 inhibitor, in patients with active ankylosing spondylitis (TORTUGA): results from a randomised, placebo-controlled, phase 2 trial. Lancet. 2018; 392(10162):2378-87). Therefore, MM provides a rapid, responsive PD measure of inflammation and will yield supportive evidence of efficacy of Compound 1.

The 12 weeks treatment duration of this trial is considered optimal to assess potential clinical benefits and improvements in spinal and sacroiliac joint inflammation, as assessed by MM. This duration is consistent with other trials of biological agents in AS (Mease. Emerging Immunomodulatory Therapies and New Treatment Paradigms for Axial Spondyloarthritis. Curr Rheumatol Rep. 2019; 21(7):35). In 24-week, placebo-controlled trials of biologics in AS, maximal clinical benefits were already apparent after 12 weeks of treatment (Davis et al. Recombinant human tumor necrosis factor receptor (etanercept) for treating ankylosing spondylitis: a randomized, controlled trial. Arthritis Rheum. 2003; 48(11):3230-6; Inman et al. Efficacy and safety of golimumab in patients with ankylosing spondylitis: results of a randomized, doubleblind, placebo-controlled, phase III trial. Arthritis Rheum 2008; 58(11):3402-12; Landewé et al. Efficacy of certolizumab pegol on signs and symptoms of axial spondyloarthritis including ankylosing spondylitis: 24-week results of a doubleblind randomised placebo-controlled Phase 3 study. Ann Rheum Dis. 2014; 73(1):39-47), suggesting that 12 weeks of treatment is the optimal duration to demonstrate the maximal benefit of an anti-inflammatory agent while minimizing the duration of treatment with placebo.

Rationale for Dose, Schedule and Regimen Selection. Daily doses of 60 mg and 150 mg Compound 1 were selected for this trial based on preclinical and clinical data in addition to simulation that supports adequate target engagement at clinically tolerated doses.

In the Phase 1 study of healthy volunteers, daily doses between 60 mg and 150 mg were associated with exposures of Compound 1 that were sufficient to achieve levels of the TE in humans that correlate with efficacy in multiple preclinical animal models. In multiple animal models of arthritis and skin inflammation, TE of 40% and above were associated with efficacy, and greater levels of TE were associated with greater improvements in disease activity. In the human leukocyte antigen—B27 transgenic (HLA-B27Tg) model of AS in rats, TE of approximately 70% was associated with a greater decrease in paw swelling as compared to therapeutic doses of etanercept, a biologic TNF-α inhibitor, and tofacitinib, a Janus kinase (JAK) inhibitor. In ex vivo studies of peripheral blood mononuclear cells (PBMCs) from healthy volunteers, TE of 60% achieved approximately 70% inhibition of TNF-α secretion. The relationship between Compound 1 exposure and TE was explored using a maximum effect (Emax) model with clinical data from the Phase 1 study. The 60 mg and 150 mg doses correspond to exposures with a predicted median TEs of approximately 67% and 79%, respectively. These two doses represent a distribution across a range of target engagement levels that can be expected to lead to clinical benefits in AS.

In a 91-day repeat-dose toxicology study with mice, the NOAEL was determined to be 100 mg/kg/day, corresponding to a mean AUCLST of 28800 ng-hr/mL for Compound 1. In humans, the highest dose tested in this study will be 150 mg, which corresponds to an observed AUCLST of 1361 ng·hr/mL for Compound 1. This represents a 21-fold margin below the mouse NOAEL for Compound 1. The NOAEL in mice was based on microscopic findings in the heart at a dose of 750 mg/kg/day (mean AUCLST of 144000 ng·hr/mL for Compound 1). This adverse finding was not observed in the 28- and 91-day studies in cynomolgus monkeys, which resulted in AUCLST at the NOAEL corresponding to the highest dose tested, 375 mg/kg/day, of 9115 for Compound 1. The exposures in monkeys represent a 6.7-fold margin for Compound 1, based on the 150-mg dose.

Safety and tolerability data from the Phase 1 study, which included single-dose and multiple-dose testing in healthy volunteers, support the proposed doses. In addition, there was limited to no observed accumulation of Compound 1 with daily dosing for 14 days. In summary, the nonclinical and clinical safety, tolerability, exposure, PD, and preclinical efficacy data indicate that the proposed doses are appropriate for this study.

Rationale for Placebo Comparator. A placebo arm is needed in this Phase 2 trial to accurately determine the benefit-risk profile of Compound 1. Placebo ASAS 20 response rates in AS clinical trials have been highly variable, ranging from 28.3% to 41.2%. The relatively high and variable placebo response rate has a significant impact on interpretation of efficacy in therapeutic AS trials and justifies the need to determine the placebo-adjusted response rate to adequately assess the treatment benefit of Compound 1 in AS.

Study Design. This is a phase 2, multicenter, randomized, double-blind, placebo-controlled, parallel-group, efficacy and safety study of two doses of Compound 1 (60 mg and 150 mg QD), compared to placebo, in subjects with active AS. The study consists of multiple periods:
Screening Period (up to 6 weeks)
Double-blind, Placebo-controlled Treatment Period (12 weeks)
Post-treatment Observational Follow-up Period (4 weeks)

Biologic-Naïve Main Study. Approximately 162 adult male and female subjects with a diagnosis of AS fulfilling the modified New York criteria for AS (van der Linden et al. Evaluation of Diagnostic Criteria for Ankylosing Spondylitis. A Proposal for Modification of the New York Criteria. Arthritis Rheum. 1984; 27(4):361-8), symptoms of active disease based on a BASDAI score ≥4, a total Back Pain Numerical Rating Scales (NRS) score ≥4, and no prior exposure to biologic treatment of AS, will be randomized 1:1:1 (54 subjects per arm) to receive either Compound 1 150 mg PO QD, Compound 1 60 mg PO QD, or matching placebo for a duration of 12 weeks. Randomization to treatment groups will be stratified by hsCRP concentration, (≤upper limit of normal/>upper limit of normal) obtained at initial Screening Visit 1. Approximately 50 additional subjects will be enrolled in a biologic-failure substudy.

Following an initial Screening at Visit 1, eligible subjects will be randomized to either Compound 1 150 mg PO QD or 60 mg PO QD or matching placebo beginning on Day 1 (Baseline Visit) of the study. Randomization and treatment assignment will be managed by an Interactive Web Response System (IWRS).

Subjects will remain in the study for a maximum of 22 weeks and will be required to attend a total of 7 study visits (from Screening Visit to Observational Follow-up Visit). Assessments for efficacy, safety, tolerability, quality of life, PK and PD will be performed at specified timepoints. Subjects who discontinue prematurely from the study at any time will be required to enter the 4-week Post-treatment Observational Follow-up Phase.

The blind will be maintained for persons responsible for the ongoing conduct of the study. Blinded persons may include but are not limited to: subjects, site personnel, Clinical Research Physician, Clinical Research Scientist, Clinical Trial Manager, Study Statistician, Data Manager, Programmers, and Clinical Research Associates.

The study will be conducted in compliance with the International Council for Harmonisation (ICH) Technical Requirements for Registration of Pharmaceuticals for Human Use/Good Clinical Practice (GCP) and applicable regulatory requirements.

Inclusion Criteria for the Biologic-naïve Main Study.

Subjects must satisfy the following criteria to be enrolled in the study:

General Patient Population:
1. Subject is ≥18 and ≤65 years of age at the time of signing the informed consent form (ICF)
2. Subject must understand and voluntarily sign an ICF prior to any study-related assessments/procedures being conducted
3. Subject is willing and able to adhere to the study visit schedule and other protocol requirements
4. Females of childbearing potential (FCBP) must:
a. Have two negative pregnancy tests as verified by the Investigator prior to starting investigational product (IP). She must agree to ongoing pregnancy testing during the course of the study, and after end of study treatment. This applies even if the subject practices true abstinence* from heterosexual contact
b. Either commit to true abstinence* from heterosexual contact (which must be reviewed on a monthly basis and source documented) or agree to use one highly effective birth control method (one that does not rely solely on hormonal contraceptives) and be able to comply with highly effective contraception without interruption, 28 days prior to starting IP, during the study therapy (including dose interruptions), and for 28 days after discontinuation of IP
c. Must not donate eggs during the study or within 28 days of the last dose of study drug Approved options for birth control are:
Any one of the following highly effective methods: intrauterine device (IUD); tubal ligation (tying your tubes); or a partner with a vasectomy
Note: Because the effect of Compound 1 on the PK of hormonal contraceptives is unknown, use of these drugs as the sole means of highly effective contraception is prohibited.
A female of childbearing potential (FCBP) is a female who: 1) has achieved menarche, 2) has not undergone a hysterectomy or bilateral oophorectomy or 3) has not been naturally postmenopausal for at least 24 consecutive months (i.e., has had menses at any time in the preceding 24 consecutive months), amenorrhea following cancer therapy does not rule out childbearing potential.
5. Male subjects must:
a. Practice true abstinence* (which must be reviewed on a monthly basis) or agree to use condoms not made out of natural [animal] membrane during sexual contact with a pregnant female or a female of childbearing potential while participating in the study, during dose interruptions and for 28 days after the last dose of investigational product, even if he has undergone a vasectomy. Any nonpregnant FCBP partner of a male subject must use an approved method of effective contraception, without interruption, during the study (including any dose interruptions) and for at least 28 days after the last dose of investigational product
* True abstinence is acceptable when this is in line with the preferred and usual lifestyle of the subject and is accepted as a contraceptive method per local guidelines or practice. Periodic abstinence (e.g., calendar, ovulation, symptothermal, postovulation methods) and withdrawal are not acceptable methods of contraception
6. Subjects agrees to limit ultraviolet (UV) exposure during the study and for at least 3 days after final dose of IP by adhering to the photoprotection guidelines below:
   a. Avoid being outdoors when the sun is at maximal intensity
   b. Wear clothing that would protect from the sun such as long-sleeves, sunglasses, and a hat
   c. Use sunscreen lotion in accordance with local guidance

Disease Specific Parameters:
7. Subject has a diagnosis of AS fulfilling the modified New York criteria for AS (van der Linden et al. Evaluation of Diagnostic Criteria for Ankylosing Spondylitis. A Proposal for Modification of the New York Criteria. Arthritis Rheum. 1984; 27(4):361-8) with radiologic entry criteria documented by central reading (historical radiographs up to 12 months old are considered acceptable)
8. Subject has active axial disease at Screening and baseline defined by a BASDAI score ≥4 (0 to 10 scale) and total back pain, as measured by a NRS ≥4 (0 to 10 scale) Prior and Current Treatment:
9. Subject must have failed prior treatment with at least 2 NSAIDs (at the maximum tolerated dose) for at least 4 weeks each, with documented inadequate response
Note: Concomitant use of one NSAID or cyclooxygenase 2 inhibitor for AS is permitted provided that the subject is receiving a stable dose 2 weeks prior to baseline and subject agrees to maintaining this dose until Week 12, unless dose is reduced, or treatment is discontinued due to safety and/or tolerability concerns.
10. Subject has never received a biologic therapy (e.g., TNF antagonist or monoclonal antibody [mAb] against IL-17A) for the treatment of AS

11. Subjects receiving statins agree to have their dose modified as per protocol requirements

Exclusion Criteria for Biologic-naïve Main Study.

Disease Specific Requirements:
1. Subject has radiographic evidence of total ankylosis of the spine
2. Subject has uncontrolled severe psoriasis (defined as Body Surface Area >10%)
3. Subject has active inflammatory bowel disease (e.g., ulcerative colitis, Crohn's disease) within 6 months of Screening Visit based on clinical assessment
4. Presence of acute anterior uveitis within 4 weeks prior to Screening Visit
5. Autoimmune diseases such as, but not limited to: systemic lupus erythematous, mixed connective tissue disease, multiple sclerosis, rheumatoid arthritis, gout, reactive arthritis, vasculitis
6. Subject has concomitant fibromyalgia, which symptoms or therapy for, in the opinion of the Investigator, will significantly impact the assessment of AS disease manifestations and activity
7. Subject has clinically significant back pain caused by diseases other than AS (e.g., degenerative disc disease, osteoarthritis) which symptoms or therapy for, in the opinion of the Investigator, will significantly impact the assessment of AS disease manifestations and activity

Prior and/or Current Medications/Therapies:
8. Concurrent treatment or treatment within the 6 months prior to Baseline with cell depleting biologic agents (e.g., anti-CD20 [e.g., rituximab], anti-CD4, anti-CD3), denosumab, anti-IL-6 [e.g., tocilizumab, sarilumab], and anti-IL-23 (e.g., ustekinumab)
9. Use of oral corticosteroids (prednisone or equivalent) >10 mg/day systemically for ≥2 weeks prior to Baseline Visit
Note: Concomitant use of prednisone ≤10 mg is permitted provided it is taken at a stable dose within 4 weeks of Baseline Visit.
10. Use of any intramuscular, intravenous or intraarticular corticosteroid treatment within 4 weeks of the Baseline Visit
11. Use of vitamin K antagonists (e.g., warfarin).
12. Treatment with isoniazid within 4 weeks of the Baseline Visit and at any time during the Screening Period, up through the first dose of IP
13. Use of any medications that are substrates of one or more of the transporters P-gp, BCRP, OCT1, OATP1B1, and OATP1B3 and have a narrow therapeutic index (e.g., methotrexate, sulfasalazine, and leflunomide).
Note: At least 1-month washout period prior to randomization is required for the conventional synthetic disease-modifying antirheumatic drugs (DMARDs), except for leflunomide, which has to be discontinued for 8 weeks prior to randomization unless a cholestyramine washout has been performed. Subjects should not discontinue any of the above synthetic DMARDS for the sole purpose of participating in this trial.
14. Participation in any study of an investigational drug within one month or 5 PK or PD half-lives of the investigational drug, whichever is longer, prior to Screening Visit, or participation in more than one study with an investigational agent for AS within one year prior to Screening Visit

General Health:
15. Major surgery performed within 8 weeks prior to Screening Visit or planned within 12 weeks after Screening Visit
16. Evidence of significant cardiac, renal, neurologic, psychiatric, endocrinologic (including uncontrolled diabetes, defined as hemoglobin A1c (HbA1c) ≥9.5%), metabolic, hepatic disease or gastrointestinal disease
17. History or evidence of congenital and/or acquired immunodeficiencies (e.g., common variable immunodeficiency, human immunodeficiency virus [HIV], etc.)
18. The subject has serologic tests during Screening consistent with infection with either hepatitis B or hepatitis C, and/or confirmed history of hepatitis B or hepatitis C infection. Subjects with isolated positive hepatitis B surface antibody are not excluded.
19. The subject has evidence on chest X-ray of lung pathology that, in the opinion of the Investigator, would pose an unacceptable safety risk in the event of further participation in the trial
Note: Examples of X-ray findings that would preclude further trial participation include active lower tract respiratory infection or suspected malignancy. If comparison with prior X-rays demonstrates unchanged findings, subject may be eligible for further participation following discussion with medical monitor.
20. History of active or latent tuberculosis (TB) infection, unless there is medical record documentation of successful completion of a standard course of treatment considered appropriate
Note: Documentation of adequate treatment for TB must be obtained and reviewed by the Medical Monitor prior to randomization. In such subjects, the QuantiFERON-TB Gold test is not needed. Instead, a chest radiograph, obtained within the 12 weeks prior to Screening or during Screening, without changes suggestive of active TB infection as determined by a qualified radiologist, is sufficient to permit further participation in the study for these subjects.
If the subject lives in, or has emigrated from, a TB high burden country (Angola, Azerbaijan, Bangladesh, Belarus, China, Democratic People's Republic of Korea, Democratic Republic of Congo, Ethiopia, India, Kazakhstan, Kenya, Kyrgyzstan, Indonesia, Moldova, Mozambique, Myanmar, Nigeria, Pakistan, Papua New Guinea, Peru, Philippines, Russian Federation, South Africa, Thailand, Ukraine, United Republic of Viet Nam, Uzbekistan, Republic of Somalia, Tajikistan, and Zimbabwe), then further study participation requires documentation of adequate treatment for either active or latent TB infection within 2 years of the Screening Visit. For subjects residing in, or emigrating from, a TB high burden country and treated for TB more than 2 years prior to the Screening Visit, further trial participation requires a negative QuantiFERON-TB Gold test during screening.
21. History of active or latent TB infection, and subject lives in, or has emigrated from, a multidrug-resistant (MDR) TB high burden country (Angola, Azerbaijan, Bangladesh, Belarus, China, Democratic People's Republic of Korea, Democratic Republic of Congo, Ethiopia, India, Kazakhstan, Kenya, Kyrgyzstan, Indonesia, Moldova, Mozambique, Myanmar, Nigeria, Pakistan, Papua New Guinea, Peru, Philippines, Russian Federation, South Africa, Thailand, Ukraine, United Republic of Viet Nam, Uzbekistan, Republic of Somalia, Tajikistan, and Zimbabwe)
22. Subject has had a household contact with a person with active TB and subject did not receive appropriate and documented prophylaxis for TB
Note: Household contact is a person who shared the same enclosed living space as the index case for one or more nights or for frequent or extended daytime periods during the 3 months before the start of current treatment
23. Active or history of recurrent bacterial, viral, fungal, mycobacterial or other infections (including, but not limited to, atypical mycobacterial disease and herpes zoster), or any major episode of infection requiring hospitalization or treatment with intravenous or oral antibiotics within 4 weeks of the Screening Visit and at any time during the Screening Period, up through the first dose of IP
24. Administration of a live or attenuated vaccine within 4 weeks prior to Baseline
25. History of malignancy (exceptions: excised and cured basal/squamous cell skin carcinomas, and cervical carcinoma in situ with no recurrence in 5 years)
26. Subject has any other significant medical/psychiatric condition or laboratory abnormality that would prevent the subject from participating in the study or places him/her at unacceptable risk for participation in the study
27. Subject with any of the following laboratory criteria:
White blood cell count (WBC)<3500/mm3 (<3.5×109/L) and >14,000/mm3 (>14×109/L)
Neutrophil count <1500/mm3 (<1.5×109/L)
Serum creatinine >1.5 mg/dL (>132.6 μmol/L)
Aspartate aminotransferase (AST) >1.5×upper limit of normal (ULN)
Alanine aminotransferase (ALT) >1.5×ULN
Total bilirubin >2×ULN
Hemoglobin <8.5 g/dL (<85 g/L)
28. Subject engages in or has a history of systemic use (e.g. smoking, ingestion) of marijuana, tetrahydrocannabinol (THC), cannabidiol (CBD oil), or cannabinoids within 4 weeks of randomization; or has a history of recreational drug abuse or significant alcohol consumption for a period of more than 3 consecutive months within 1 year prior to Screening. Significant alcohol consumption is defined as more than 14 oz (420 mL) per week in females and more than 21 oz (630 mL) per week in males, on average (1 oz/30 mL of alcohol is present in one 12 oz/360 mL beer, one 4 oz/120 mL glass of wine, or a 1 oz/30 mL measure of 40% proof alcohol). Subjects must also agree not to engage in recreational drug abuse, significant alcohol consumption as described above, or systemic use of marijuana, THC, CBD oil, or cannabinoids for the duration of the study.
29. Subject has history of drug-related photosensitivity
30. Subject has a known hypersensitivity to Compound 1 or any ingredient in the investigational product

Biologic-Failure Substudy. Approximately 50 subjects with AS who have failed not more than 1 biologic agent taken for AS due to inadequate response to an approved dose for at least 12 weeks and/or unacceptable safety/tolerability with at least one dose of a biologic agent (in the opinion of the Investigator) will be recruited into a separate substudy, conducted concurrently with the biologic-naïve main study. A minimum of 50% of biologic failure subjects will be recruited due to inadequate response to treatment. Subjects will be randomized with 2:2:1 ratio to have 20 subjects each to receive either treatment with Compound 1 150 mg PO QD, Compound 1 60 mg PO QD, and 10 subjects to receive matching placebo, for a duration of 12 weeks. Subjects will remain in the study for a maximum of 22 weeks and will be required to attend a total of 7 study visits (from Screening Visit to Observational Follow-up Visit). Subjects who discontinue prematurely from the study at any time will be required to enter the 4-week Post-treatment Observational Follow-up Phase.

Inclusion Criteria for Biologic-Failure Substudy. Subjects participating in the biologic-failure substudy must:
1. Meet all the inclusion criteria for subjects in the biologic-naïve main study except Inclusion Criterion 10
2. Have discontinued one and only one biologic (e.g., TNF antagonist or monoclonal antibody [mAb] against IL-17A) for AS, either due to inadequate response to an approved biologic dose for at least 12 weeks and/or unacceptable safety/tolerability with at least one dose of a biologic agent (in the opinion of the Investigator). The following minimum washout periods prior to Baseline must be adhered to for the biologics or their corresponding biosimilars:

a. etanercept: 4 weeks

b. adalimumab, certolizumab pegol, golimumab, infliximab: 8 weeks

c. secukinumab, ixekizumab: 12 weeks
Note: Any questions regarding washout periods prior to baseline should be directed to the Medical Monitor. Subjects should not discontinue any of the above biologics for the sole purpose of participating in this trial

Exclusion Criteria for Biologic-Failure Substudy. Subjects participating in the biologic-failure substudy must:

1. Meet all the exclusion criteria listed for subjects in the biologic-naïve main study

Efficacy Assessment. The following sections describe the efficacy measures and assessments obtained or completed directly from the subject, or efficacy evaluations completed by the Investigator (or authorized site staff member) on the subject.

Assessment of SpondyloArthritis International Society Response Criteria. The ASAS 20 and ASAS 40 are validated response criteria widely used in the evaluation of efficacy of agents used in the treatment of axSpA (Sieper, et al. The Assessment of SpondyloArthritis international Society (ASAS) handbook: a guide to assess spondyloarthritis. Ann Rheum Dis 2009; 68(Suppl 2); iil-ii44). The ASAS 20 is defined as improvement ≥20% and ≥1 unit on a scale of 0 to 10 in each of the 3 domains, with no worsening in the fourth, where the domains are physical function, total back pain, patient global assessment of disease, and inflammation (mean of BASDAI NRS Questions #5 and #6 for morning stiffness) (Anderson et al. Ankylosing spondylitis assessment group preliminary definition of short-term improvement in ankylosing spondylitis. Arthritis Rheum 2001; 44(8):1876-86).

The ASAS 40 is defined as improvement ≥40% and ≥2 units on a scale of 0 to 10 in each of the 3 domains, with no worsening in the fourth, where the domains are physical function, total back pain, patient global assessment of disease, and inflammation (mean of BASDAI NRS Questions #5 and #6 for morning stiffness) (Brandt, et al. Development and preselection of criteria for short term improvement after anti-TNF alpha treatment in ankylosing spondylitis. Ann Rheum Dis 2004; 63 (11): 1438-44).

Ankylosing Spondylitis Disease Activity Score with CRP. The ASDAS-CRP is a validated disease activity index in AS that combines patient reported assessments of back pain (BASDAI question 2), duration of morning stiffness (BASDAI question 6), peripheral joint pain and/or swelling (BASDAI question 3), general wellbeing, and CRP, in a weighted manner (van der Heijde, et al. ASDAS, a highly discriminatory ASAS-endorsed disease activity score in patients with ankylosing spondylitis. Ann Rheum Dis. 2009; 68(12):1811-18). The cut-off values for disease activity states and improvement scores are defined as follows: <1.3 inactive disease, ≥1.3 and <2.1 low disease activity, ≥2.1 and ≤3.5 high disease activity and, 3.5 very high disease activity. The minimum clinically important difference (MCID), are defined as: change of at least 1.1 unit for ‘clinically important improvement’ and change of at least 2.0 units for ‘major improvement’ (Machado et al. Ankylosing Spondylitis Disease Activity Score (ASDAS): defining cut-off values for disease activity states and improvement scores. Ann Rheum Dis. 2011; 70(1):47-53; Machado et al. Ankylosing Spondylitis Disease Activity Score (ASDAS): 2018 update of the nomenclature for disease activity states. Ann Rheum Dis. 2018; 77(10):1539-40).

Bath Ankylosing Spondylitis Disease Activity Index. The BASDAI is a composite score based on a subject self-administered survey of six questions using a 0 to 10 unit numerical rating scale (NRS) that assesses the subject's five major symptoms of AS: 1) fatigue; 2) spinal pain; 3) peripheral joint pain/swelling; 4) areas of localized tenderness; 5a) morning stiffness severity upon wakening; 5b) morning stiffness duration upon wakening (Calin et al. Defining disease activity in ankylosing spondylitis: is a combination of variables (Bath Ankylosing Spondylitis Disease Activity Index) an appropriate instrument? Rheumatology (Oxford). 1999; 38(9): 878-82; Sieper, et al. The Assessment of SpondyloArthritis international Society (ASAS) handbook: a guide to assess spondyloarthritis. Ann Rheum Dis 2009; 68(Suppl 2); ii1-ii44). The subject will be asked to mark the box with an X on a 0 to 10 unit NRS for each of the 6 questions. To give each of the five symptoms equal weighting, the mean of the two scores relating to morning stiffness is taken. The resulting 0 to 50 score is divided by 5 to give a final 0 to 10 BASDAI score. A BASDAI score of 4 or greater is considered to be indicative of active AS disease.

Bath Ankylosing Spondylitis Functional Index. The BASFI is a composite score based on a subject self-administered survey of ten questions using a 0 to 10 unit numerical rating scale (NRS) that assesses a subject's degree of mobility and functional ability (Calin et al. A new approach to defining functional ability on ankylosing spondylitis: the development of the Bath Ankylosing Spondylitis Functional Index. J Rheumatol. 1994; 21(12):2281-5; Sieper, et al. The Assessment of SpondyloArthritis international Society (ASAS) handbook: a guide to assess spondyloarthritis. Ann Rheum Dis 2009; 68(Suppl 2); iil-ii44). The questionnaire consists of eight questions regarding function in AS and the two last questions reflecting the subject's ability to cope with everyday life. The subject will be asked to mark the box with an X on a 0 to 10 unit NRS for each of the 10 questions, on which the left-hand box of 0 represents “easy,” and the right-hand box represents impossible.” The resulting 0 to 100 score is divided by 10 to give a final 0 to 10 BASFI score. A higher BASFI score correlates to reduced functional ability.

Patient Global Assessment of Disease Activity. The Patient Global Assessment of Disease Activity is the subject's assessment of how active their spondylitis was on average during the last week. The subject will be asked to mark the box with an X on a 0 to 10 unit NRS in which the left-hand box of 0 represents “not active” and the right-hand box represents “very active” (Sieper, et al. The Assessment of SpondyloArthritis international Society (ASAS) handbook: a guide to assess spondyloarthritis. Ann Rheum Dis 2009; 68(Suppl 2); iil-ii44).

Total Back Pain. they have in their spine due to AS. The subject will be asked to mark the box with an X on a 0 to 10 unit NRS in which the left-hand box of 0 represents “no pain” and the right-hand box represents “most severe pain” (Sieper, et al. The Assessment of SpondyloArthritis international Society (ASAS) handbook: a guide to assess spondyloarthritis. Ann Rheum Dis 2009; 68(Suppl 2); ii1-ii44).

Night Time Back Pain. The night time back pain NRS is the subject's assessment of, on average last week, how much pain they have in their spine due to AS at night. The subject will be asked to mark the box with an X on a 0 to 10 unit NRS in which the left-hand box of 0 represents “no pain” and the righthand box represents “most severe pain” (Sieper, et al. The Assessment of SpondyloArthritis international Society (ASAS) handbook: a guide to assess spondyloarthritis. Ann Rheum Dis 2009; 68(Suppl 2); ii1-ii44).

Bath Ankylosing Spondylitis Metrology Index-Linear. The BASMI-Linear was designed to assess axial status (ie, cervical, dorsal and lumbar spine, hips, and pelvic soft tissue) and to define clinically significant changes in spinal movement (Jenkinson, et al. Defining Spinal Mobility in Ankylosing Spondylitis (AS): The Bath AS Metrology Index. J Rheumatol 1994; 21(9):1694-8; Sieper, et al. The Assessment of SpondyloArthritis international Society (ASAS) handbook: a guide to assess spondyloarthritis. Ann Rheum Dis 2009; 68(Suppl 2); ii1-ii44). Five dimensions of movement (lateral lumbar flexion, tragus to wall, forward lumbar flexion, maximal intermalleolar distance, and cervical rotation) are measured and normalized on 0 to 10 unit NRS. The average of these scores is the total BASMI-Linear score, with a higher value indicating more severe limitation in spinal mobility.

Occiput to Wall Measurement. Occiput to wall measurement is the distance measured between the occiput located on the back of the subject's skull and the wall. The subject stands with heels and shoulder against the wall with the back straight. The chin is at the usual carry level. The maximal effort to touch the head against the wall is asked of the subject. The distance between the occiput and the wall is measured in centimeters (cm) (Sieper, et al. The Assessment of SpondyloArthritis international Society (ASAS) handbook: a guide to assess spondyloarthritis. Ann Rheum Dis 2009; 68(Suppl 2); ii1-ii44).

Chest Expansion Measurement. When ankylosing spondylitis affects the mid-back region (thoracic spine), normal chest expansion may be compromised. The chest expansion measurement is the difference between the circumference of the chest in maximal inspiration and maximal expiration. To conduct the test, the subject has his/her hands resting on or behind the head. The amount of chest expansion is measured from deep expiration to full inspiration and is measured at the level of the fourth intercostal space anteriorly in males and just below the breasts in females. The difference between the maximal inspiration and expiration is recorded in centimeters (cm) (Sieper, et al. The Assessment of SpondyloArthritis international Society (ASAS) handbook: a guide to assess spondyloarthritis. Ann Rheum Dis 2009; 68(Suppl 2); ii1-ii44).

Enthesitis Evaluation. Enthesitis, or the swelling of the sites where tendons or ligaments insert into the bone, is a prominent clinical manifestation in subjects with AS. The Maastricht Ankylosing Spondylitis Enthesitis Score (MASES) will be used in this study to measure the severity of a subject's enthesitis (Heuft-Dorenbosch, et al. Assessment of enthesitis in ankylosing spondylitis. Ann Rheum Dis. 2003; 62(2):127-32).

Peripheral Joint Count. An ASAS joint evaluation which includes the “44 tender and 44 swollen” joint counts (Sieper, et al. The Assessment of SpondyloArthritis international Society (ASAS) handbook: a guide to assess spondyloarthritis. Ann Rheum Dis 2009; 68(Suppl 2); iil-ii44) will be performed on all subjects to monitor peripheral joint involvement. Non weighted measures will be used to assess tender and swollen joints. In order to maintain consistency throughout the study, preferably the same evaluator should perform the peripheral joint assessments at the study site at each study visit.

MRI of Sacroiliac Joint and Spine (SPARCC Method). The MM assessments will be conducted at all sites. Details regarding the MM specific procedures and the consistent acquisition of MRI images will be outlined in a separate image acquisition document.

MM assessment will be performed on the sacroiliac joints and the entire spine (cervical, thoracic, and lumbar). The baseline MRI must be performed between Visits 1 and 2 inclusive. Subsequent MRIs should be completed at Visit 6 (the final treatment visit) or at the Early Termination Visit. If the Early Termination Visit occurs before Week 6, then MRI of sacroiliac joint should not be done.

All MM visits should be scheduled well in advance to allow for proper planning. In addition, it is strongly recommended to schedule two MRI visits (an initial and a repeat visit) for each MRI time point in order to be assured of a high-quality MM scan at each protocol-specified time point. The initial and repeat MRI visits should be scheduled approximately 7 to 14 days apart to allow enough time for confirmation of quality scans from the initial MM. If the initial MRI scans are of acceptable quality, then the scheduled repeat MRI session can be cancelled. The scoring of the standardized Mills will be conducted by well-trained, independent, central readers.

Subject Reported Outcomes.

Ankylosing Spondylitis Quality of Life. The ASQoL is a validated disease specific patient reported outcomes instrument to assess the impact of ankylosing spondylitis (AS) on the quality of life of individuals with emphasis on the ability of the person to fulfill his or her needs (Doward, et al. Development of the ASQoL: a quality of life instrument specific to ankylosing spondylitis. Ann Rheum Dis. 2003; 62(1):20-6). It consists of 18 items requesting a yes (score=1) or no (score=0) response to questions related to the impact of pain on sleep, mood, motivation, ability to cope, activities of daily living (ADL), independence, relationships, and social life. The summary score ranges 0-18 with higher scores indicating worse quality of life. The MCID was defined as a 1.8-point change for ASQoL (van der Heijde, et al. 2007a).

Medical Outcome Study Short Form 36-Item Health Survey, Version 2. The SF-36 (Ware, et al. The MOS 36-item short-form health survey (SF-36). I. Conceptual framework and item selection. Med Care. 1992; 30(6):473-83) is a validated, self-administered 36-item general health status instrument often used in clinical trials and health services research. It consists of 8 scales: physical function (PF), role limitations-physical (RP), vitality (VT), general health perceptions (GH), bodily pain (BP), social function (SF), role limitations-emotional (RE), and mental health (MH) (Ware, et al. The MOS 36-item short-form health survey (SF-36). I. Conceptual framework and item selection. Med Care. 1992; 30(6):473-83). Scale scores range from 0 to 100, with higher scores indicating better health. Two overall summary scores can also be obtained—a Physical Component Summary score (PCS) and a Mental Component Summary score (MCS). The PCS and MCS scores are transformed to have a mean of 50 and standard deviation of 10, with higher scores indicating better health. The concepts measured by the SF-36 are not specific to any age, disease, or treatment group, allowing comparison of relative burden of different diseases and the relative benefit of different treatments. Version 2 of the SF-36 will be used in this study.

ASAS Health Index. The ASAS Health Index is a validated linear composite measure and includes 17 items and is intended to capture relevant information on functioning and health of patients with AS (Kiltz, et al. The ASAS Health Index (ASAS HI)—a new tool to assess the health status of patients with spondyloarthritis. Clin Exp Rheumatol 2014; 32 (5 Suppl. 85):S105-S108).

Permitted Concomitant Medications and Procedures. Concomitant use of one NSAID or cyclooxygenase (COX)-2 inhibitor on a regular basis up to the maximum recommended dose as per local guidelines is permitted, provided the drug is for treatment of AS and is used at a stable dose for at least 2 weeks prior to baseline (Day 1 of study). The dose and frequency of the medication must be maintained stable until conclusion of study treatment (end of Week 12), even if subject experiences improvement in disease activity. If the subject experiences an adverse event (e.g., gastrointestinal bleed) then dose reduction, stopping or switching to another NSAID or COX-2 inhibitor, or discontinuation of the agent is allowed. As needed use of NSAIDs or COX-2 inhibitors is not recommended during the study and is not permitted within the 24 hours prior to a study visit.

In case the subject requires as needed medication for pain, acetaminophen/paracetamol and/or low-strength opioid analgesics up to the maximum recommended doses per local guidelines may be used but cannot be taken within 24 hours prior to a study visit with disease activity assessment.

Acetylsalicylic acid at a dose of <325 mg/day for cardiac prophylaxis is allowed.

Bisphosphonate therapy is allowed if stable for at least 1 year prior to randomization and must be maintained stable until conclusion of study treatment (end of Week 12).

Concomitant use of systemic corticosteroids at or up to 10 mg/day of prednisone or equivalent is permitted, provided the drug is used at a stable dose for at least 4 weeks prior to baseline. The dose and frequency of the medication must be maintained stable until conclusion of study treatment (end of Week 12).

Subjects may receive statins (3-hydroxy-3-methyl-glutaryl-coenzyme A reductase [HMG-CoA reductase] inhibitors) during the study; however, this will require careful monitoring. To ensure the safety of the subjects, statins will be dose adjusted to an appropriate therapeutic level while being taken during the study.

Permitted Medications That Require Careful Monitoring. Drug-drug interactions involving Compound 1 have not been studied in humans. In vitro assessments suggest that Compound 1 has the potential for direct and time-dependent inhibition of intestinal CYP3A4/5. Subjects receiving drugs that are eliminated through this enzyme pathway should be closely monitored during co-administration.

Compound 1 may potentially inhibit the transporters P-gp, BCRP, OATP1B1, OATP1B3 and OCTs. Subjects receiving drugs that are substrates of these transporters should be closely monitored for potential toxicities during participation in the study. A partial list of substrates of these transporters, including substrates that are therapeutically relevant to common comorbidities in the AS population, include methotrexate, sulfasalazine, leflunomide, rosuvastatin, aliskiren, ambrisentan, colchicine, cyclosporine, dabigatran etexilate, digoxin, everolimus, fexofenadine, methotrexate, ranolazine, rivaroxaban, saxagliptin, sirolimus, sitagliptin, talinolol, ticagrelor, tolvaptan, ambrisentan, atorvastatin, ezetimibe, fluvastatin, glyburide, rosuvastatin, simvastatin acid, pitavastatin, pravastatin, repaglinide, telmisartan, valsartan, olmesartan, mycophenolic acid, metformin, gabapentin, pramipexole, tramadol, varenicline. Questions regarding potential co-administration of Compound 1 with drugs that are substrates of CYP3A4/5 or these transporters should be discussed with the medical monitor.

Statins. Due to potential inhibition of CYP3A4, OATP1B1, OATP1B3, BCRP and P-gp by Compound 1, the following must be done for subjects receiving statins during the study:

All subjects receiving statins should be prescribed the lowest approved statin dose for adults approximately 1 week before the Baseline Visit. For example, a subject receiving atorvastatin 20 mg during the Screening Period should be instructed to begin taking atorvastatin 10 mg, instead, approximately 1 week prior to randomization.

Note: Subjects who are already receiving the lowest approved statin dose for adults should remain on that dose.

At post-randomization study visits. based on results of the subjects' lipid profile, the statin dose may be increased to the next highest approved dose, if medically indicated.

Note: Investigators should monitor closely for AEs associated with increased statin exposures (e.g., myalgia, liver function test abnormalities), and follow the warnings and monitoring recommendations of the respective statin label.

Metformin. As a result of potential inhibition of OCT proteins by Compound 1, the following must be done for subjects receiving metformin during the study:

During the Screening period of the study, all subjects receiving metformin should have serum glucose or HbA1c assessment to determine if they are at goal for glycemic control per local clinical guidelines.

Subjects at glycemic control goal and on a glucose-lowering regimen that includes no greater than metformin 500 mg per day may proceed to randomization in the study.

At Visit 5, the serum blood glucose profile will be assessed. Based on results of the subjects' glycemic control profile at the third visit, a non-metformin glucose-lowering agent may be added, if medically indicated, provided it is not a prohibited concomitant medication.

Note: Following randomization, Investigators should monitor subjects closely for AEs associated with increased metformin exposures (eg, gastrointestinal symptoms, such as diarrhea, nausea, vomiting, flatulence; lactic acidosis) and follow the warnings and recommendations in the metformin label. Thus, more frequent assessment of the anion gap and renal function from the serum chemistry values may be warranted.

Prohibited Concomitant Medications. The following medications cannot be administered for the specified times prior to the initiation of study IP and for the duration of the study:
Use of Vitamin K antagonists (e.g., warfarin) during the study.
Use of methotrexate, sulfasalazine or leflunomide.
Use of any medications that are substrates of the transporters P-gp, BCRP, OATP1B1, OATP1B3, OCTs and with a narrow therapeutic index. Examples include digoxin, cyclosporine, leflunomide, mycophenolic acid, procainamide, sirolimus, everolimus, and dabigatran etexilate.
Note: Drugs considered to be substrates of these transporters and without a narrow therapeutic index (e.g., OATP1B1/3 substrate—statins, and OCTs substrate—metformin should be closely monitored for potential drug interactions while subjects are participating in the study. Additional examples of substrates of these transporters include methotrexate, sulfasalazine, leflunomide, rosuvastatin, aliskiren, ambrisentan, colchicine, cyclosporine, dabigatran etexilate, digoxin, everolimus, fexofenadine, methotrexate, ranolazine, rivaroxaban, saxagliptin, sirolimus, sitagliptin, talinolol, ticagrelor, tolvaptan, ambrisentan, atorvastatin, ezetimibe, fluvastatin, glyburide, rosuvastatin, simvastatin acid, pitavastatin, pravastatin, repaglinide, telmisartan, valsartan, olmesartan, mycophenolic acid, metformin, gabapentin, pramipexole, tramadol, varenicline.
Use of high potency opioid analgesics (e.g., methadone, hydromorphone, morphine or oxycodone).
Treatment with any biologic indicated for AS (e.g., anti-TNF or anti-IL-17A mAb) Introduction of a JAK inhibitor or immunomodulating therapy including but not limited to 6-mercaptopurine, azathioprine, cyclosporine or other calcineurin inhibitors (eg, sirolimus, tacrolimus), gold therapies.
Treatment with any systemic, intravenous, intramuscular or intra-articular corticosteroid. Use of any other investigational drug.

All publications mentioned herein are hereby incorporated by reference in their entirety.

We claim:

1. A method of treating, stabilizing or lessening the severity or progression of one or more diseases or disorders associated with MK2, wherein the method comprises administering to a patient in need thereof a pharmaceutically acceptable composition comprising Compound 1: 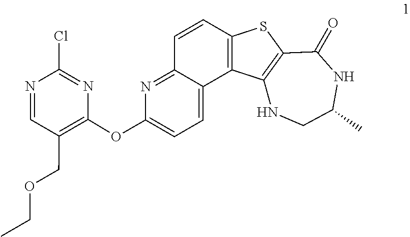 [SMILES: CCOCc1cnc(Cl)nc1Oc1ccc2c(ccc3sc4c(c32)NC[C@@H](C)NC4=O)n1]
or a pharmaceutically acceptable salt thereof.

2. The method of claim 1, wherein the disease or disorder associated with MK2 is selected from an autoimmune disorder, a chronic inflammatory disorder, an acute inflammatory disorder, or an auto-inflammatory disorder.

3. The method of claim 2, wherein the disease or disorder associated with MK2 is selected from rheumatoid arthritis, psoriatic arthritis, psoriasis, and ankylosing spondylitis.

4. The method of any of claims 1-3, wherein Compound 1 is administered once a day (“QD”).

5. The method of any of claims 1-4, wherein Compound 1 is administered in an amount of about 3 mg to about 1000 mg.

6. The method of any of claims 1-5, wherein Compound 1 is administered in an amount of about 3 mg to about 15 mg, about 10 mg to about 25 mg, about 15 mg to about 50 mg, about 25 mg to about 75 mg, about 50 mg to about 100 mg, about 75 mg to about 125 mg, about 100 mg to about 150 mg, or about 125 mg to about 200 mg.

7. The method of any of claims 1-6, wherein Compound 1 is administered in a unit dose comprising about 3 mg, about 5 mg, about 10 mg, about 15 mg, about 25 mg, about 50 mg, about 75 mg, or about 100 mg of Compound 1.

8. The method of any of claim 1-7, wherein Compound 1 is administered in an oral dosage form.

9. The method of claim 8, wherein the oral dosage form is a capsule.

10. The method of claim 8 or claim 9, wherein Compound 1 is administered in a spray-dried dispersion formulation.

11. The method of claim 10, wherein the spray-dried dispersion formulation comprises Compound 1, HPMCAS, microcrystalline cellulose, croscarmellose sodium, silicon dioxide, and magnesium stearate.
